(19) United States
(12) Patent Application Publication
(10) Pub. No.: US 2026/0345316 A1
(43) Pub. Date: Nov. 5, 2026

(54) OCULAR IMPLANT

(71) Applicant: BEAUEYEVISION CO., LTD, Uiwang-si, Gyeonggi-do (KR)

(72) Inventor: Dong Ho LEE, Seoul (KR)

(21) Appl. No.: 19/486,589

(22) PCT Filed: May 21, 2024
(86) PCT No.: PCT/KR2024/006877

§ 371 Date: Nov. 21, 2025

(30) Foreign Application Priority Data
Jun. 27, 2023 (KR) 10-2023-0082260
Jun. 27, 2023 (KR) 10-2023-0082261
Jun. 27, 2023 (KR) 10-2023-0082262
Jun. 27, 2023 (KR) 10-2023-0082263
Jun. 27, 2023 (KR) 10-2023-0082264
Feb. 27, 2024 (KR) 10-2024-0028124
Feb. 27, 2024 (KR) 10-2024-0028125

(51) Int. Cl. A61F 2/14; A61B 90/00; A61F 2/00

(52) U.S. Cl. CPC A61F 2/147; A61B 90/02; A61F 2002/0081; A61F 2210/0057; A61F 2230/0006; A61F 2230/0008; A61F 2250/0007; A61F 2250/0036

(57) ABSTRACT

The present invention discloses an ocular implant. According to the present invention, there is provided an ocular implant that can be permanently implanted on the eyeball without the inconvenience of attachment or detachment and that forms an aesthetically pleasing appearance integrated with the optically visible iris-pupil complex without blocking the light entrance aperture.

### TECHNICAL FIELD

The present invention relates to an ocular implant.

### BACKGROUND ART

Circle color lenses used for cosmetic purposes to enlarge the appearance of the optically visible iris-pupil complex are formed by inserting a dye between two lenses. Compared to contact lenses for vision correction, circle lenses have reduced oxygen permeability and an uneven surface. As a result, the contact area and opportunities for bacteria to come into contact with the circle lens increase, which may cause adverse effects such as neovascularization, keratitis, corneal ulcer, and corneal edema. For example, neovascularization may reduce the apparent size of the optically visible iris-pupil complex, which is contrary to the cosmetic purpose of the circle lens. In addition, corneal hypoxia may induce neovascularization, thereby causing whitening or clouding of the peripheral cornea.

### SUMMARY OF INVENTION

### Problems to be Solved by Invention

An object of the present invention is to provide an ocular implant that can be permanently implanted on the eyeball without the inconvenience of attachment or detachment, and that can form an aesthetically pleasing appearance integrated with the optically visible iris-pupil complex without blocking the light entrance aperture.

Another object of the present invention is to provide an ocular implant that closely adheres to an irregular edge of the eyeball so as to minimize side effects while improving ease of handling.

Still another object of the present invention is to provide an ocular implant that can be adaptively deformed to optimize the shape for various optically visible iris-pupil complex shapes and sizes of respective implant recipients, while reducing manufacturing costs through a single-design mold, thereby providing versatility.

Further another object of the present invention is to provide an ocular implant that can improve the convenience of surgical procedures while preventing side effects that may result from a structure designed for surgical convenience.

### Means for Solving Problems

An ocular implant according to one embodiment of the present invention may be permanently implanted on the eyeball of an implant recipient to provide an enlarged appearance of the implant recipient's optically visible iris-pupil complex,
the ocular implant may include:
   a central opening configured to receive light incident on the pupil that provides an optical aperture of the eyeball;
   an inner annular edge surrounding and defining the opening;
   an outer annular edge disposed opposite the inner annular edge that defines the opening and surrounds the opening together with the inner annular edge; and
   an optically visible iris-pupil complex expansion part formed with a variable thickness that changes from the inner annular edge to the outer annular edge between the inner annular edge and the outer annular edge.

In addition, an ocular implant according to one embodiment of the present invention may be permanently implanted on the eyeball of an implant recipient to provide an enlarged appearance of the implant recipient's optically visible iris-pupil complex,
the ocular implant may include:
   a central opening configured to receive light incident on the pupil that provides an optical aperture of the eyeball;
   an inner annular edge surrounding and defining the opening;
   an outer annular edge disposed opposite the inner annular edge that defines the opening and surrounds the opening together with the inner annular edge; and
   an adaptive stretchable part configured to provide adaptive longitudinal stretching along the perimeter of at least one of the inner annular edge and the outer annular edge.

Further, an ocular implant according to one embodiment of the present invention may be permanently implanted on the eyeball of an implant recipient to provide an enlarged appearance of the implant recipient's optically visible iris-pupil complex,
the ocular implant may include:
   a central opening configured to receive light incident on the pupil that provides an optical aperture of the eyeball;
   an inner annular edge surrounding and defining the opening;
   an outer annular edge disposed opposite the inner annular edge that defines the opening and surrounds the opening together with the inner annular edge; and
   an optically visible iris-pupil complex expansion part formed between the inner annular edge and the outer annular edge, the optically visible iris-pupil complex expansion part including first and second ends that are separated from each other through an incision and are joined to each other through respective surgical holes formed therein.

Furthermore, an ocular implant according to one embodiment of the present invention is permanently implanted on the eyeball of an implant recipient to provide an enlarged appearance of the implant recipient's optically visible iris-pupil complex,
the ocular implant may include:
   an eyeball expansion part including a central opening configured to receive light incident on the pupil that provides an optical aperture of the eyeball;
   an inner annular edge surrounding and defining the opening;
   an outer annular edge disposed opposite the inner annular edge that defines the opening and surrounds the opening together with the inner annular edge; and
   an optically visible iris-pupil complex expansion part formed between the inner annular edge and the outer annular edge, the optically visible iris-pupil complex expansion part including first and second ends that are separated from each other through an incision and form a catch-engaging or hook-engaging connection with each other.

### Advantageous Effects

According to the present invention, there is provided an ocular implant that can be permanently implanted on the eyeball without the inconvenience of attachment or detachment, and that can form an aesthetically pleasing appearance integrated with the optically visible iris-pupil complex without blocking the light entrance aperture.

According to the present invention, there is provided an ocular implant that closely adheres to an irregular edge of the eyeball so as to minimize side effects while improving ease of handling.

According to the present invention, there is provided an ocular implant that can be adaptively deformed to optimize the shape for various optically visible iris-pupil complex shapes and sizes of respective implant recipients, while reducing manufacturing costs through a single-design mold, thereby providing versatility.

According to the present invention, there is provided an ocular implant that can improve the convenience of surgical procedures while preventing side effects that may result from a structure designed for surgical convenience.

### BRIEF DESCRIPTION OF DRAWINGS

FIG. 1 is a schematic view illustrating the structure of an eyeball into which an ocular implant according to one embodiment of the present invention is permanently implanted.

FIG. 2 is a view describing a procedure for implanting an ocular implant according to one embodiment of the present invention, which shows an incision formed at a selected location along an outer ring of the optically visible iris-pupil complex.

FIG. 3 is a perspective view illustrating an ocular implant according to one embodiment of the present invention.

FIG. 4 is a plan view illustrating the ocular implant shown in FIG. 3 as viewed from the front.

FIG. 5 is a cross-sectional view taken along line V-V′ of FIG. 3.

FIG. 6 is a perspective view describing an adaptive stretchable part applied to the ocular implant according to one embodiment of the present invention.

FIG. 7 is a perspective view describing a modified example of the adaptive stretchable part shown in FIG. 6, applied to the ocular implant according to one embodiment of the present invention.

FIGS. 8A and 8B are cross-sectional views of the ocular implant taken along a circumferential direction of the optically visible iris-pupil complex expansion part, which describe different roughened surfaces having isotropic and anisotropic rotational resistance, respectively.

FIG. 9 is a plan view of the ocular implant, taken from a frontal direction, illustrating surgical holes applied to the ocular implant according to one embodiment of the present invention.

FIGS. 10A to 10C are perspective views illustrating catch-engaging or hook-engaging connections between first and second slits applied to ocular implants according to different embodiments of the present invention.

FIGS. 11A and 11B are views illustrating catch-engaging or hook-engaging connections between assembly slits and assembly holes applied to the ocular implant according to one embodiment of the present invention.

FIG. 12 is a view illustrating a modified embodiment of the ocular implant illustrated in FIG. 4.

FIG. 13 is a view illustrating a modified embodiment of the ocular implant illustrated in FIG. 6.

FIG. 14 is a view illustrating a composite configuration of different mechanisms that connect first and second ends separated from each other through an incision in one embodiment of the present invention.

FIG. 15 is a view illustrating a modified embodiment of the ocular implant illustrated in FIG. 9.

### BEST MODE FOR CARRYING OUT INVENTION

An ocular implant according to one embodiment of the present invention is permanently implanted on the eyeball of an implant recipient to provide an enlarged appearance of the implant recipient's optically visible iris-pupil complex,
the ocular implant may include:
   a central opening configured to receive light incident on the pupil that provides an optical aperture of the eyeball;
   an inner annular edge surrounding and defining the opening;
   an outer annular edge disposed opposite the inner annular edge that defines the opening and surrounds the opening together with the inner annular edge; and
   an optically visible iris-pupil complex expansion part formed with a variable thickness that changes from the inner annular edge to the outer annular edge between the inner annular edge and the outer annular edge.

For example, the inner annular edge and the outer annular edge may surround the opening along different contours.

For example, the inner annular edge may be formed in an oval shape along the contour of the optically visible iris-pupil complex that forms the light entrance aperture, so as not to cover the optically visible iris-pupil complex but to provide an appearance integrated with the optically visible iris-pupil complex. The inner annular edge may be shaped to surround the outer rim of the optically visible iris-pupil complex at an adjacent position.

The outer annular edge may be formed in a circular shape to provide an aesthetically circular appearance.

For example, the inner annular edge may be formed in an oval shape having different major axis length and minor axis length.

The major axis may extend in a direction in which the implant recipient's pair eyes face each other.

The minor axis may extend in a direction perpendicular to the major axis.

For example, the optically visible iris-pupil complex expansion part between the inner annular edge and the outer annular edge may be formed such that one position along the minor axis of the inner annular edge has a greater width than the opposite position.

To this end, the oval shape of the inner annular edge may be positioned in an offset manner within the circular shape of the outer annular edge, biased toward the opposite side.

For example, the optically visible iris-pupil complex expansion part may include:
a posterior inclined surface disposed to face the eyeball so as to closely adhere to the eyeball; and
an anterior curved surface disposed toward an external space opposite to the eyeball.

For example, the inner annular edge and the outer annular edge may each form an anterior portion of the eyeball and may appear as an ellipse and a circle when viewed along a frontal direction from the anterior portion of the eyeball, where a crystalline lens that performs a lens function and a pupil forming a light entrance aperture are located, toward a posterior portion of the eyeball.

The posterior inclined surface may be formed at a constant tilting angle from a vertical plane perpendicular to the frontal direction, such that the inner annular edge and the outer annular edge define an anterior position and a posterior position, respectively.

For example, the posterior inclined surface may provide a support surface that is supported by an inclined surface formed in a packaging container of the ocular implant.

For example, the anterior curved surface may be formed along a spline curve whose curvature or radius of curvature varies along the inclination direction of the posterior inclined surface.

For example, the optically visible iris-pupil complex expansion part may have a variable thickness such that the thickness between the posterior inclined surface and the anterior curved surface varies.

For example, the thickness of the optically visible iris-pupil complex expansion part may be measured along a direction perpendicular to the posterior inclined surface, from the posterior inclined surface to the anterior curved surface.

For example, the inner annular edge may be formed by contact between the anterior curved surface and the posterior inclined surface, which define the thickness of the optically visible iris-pupil complex expansion part, at a position adjacent to the opening.

For example, the outer annular edge may be formed by contact between the anterior curved surface and the posterior inclined surface, which define the thickness of the optically visible iris-pupil complex expansion part, at a position opposite to the inner annular edge defining the opening.

For example, the anterior curved surface and the posterior inclined surface, which define the thickness of the optically visible iris-pupil complex expansion part, may contact each other to form the inner annular edge and the outer annular edge, each having a rounded edge shape.

For example, the inner annular edge may be formed with a relatively gently rounded edge shape.

The outer annular edge may be formed with a relatively sharply rounded edge shape.

For example, the thickness at the edge of the inner annular edge may be smaller than the thickness at the edge of the outer annular edge.

For example, the inner annular edge may extend to an irregular edge between a cornea having a large-curvature portion that convexly protrudes forward and a sclera having a small-curvature portion extending posteriorly from the cornea, among the membrane tissues surrounding the entire eyeball to maintain its shape, and the inner annular edge may have a relatively gently rounded edge shape that defines one side of the ocular implant.

The outer annular edge, which closely adheres to the sclera in the large-curvature portion, may have a relatively sharply rounded edge shape that defines the opposite side of the ocular implant, so as to prevent lifting from the sclera or the formation of a gap between the sclera and the outer annular edge.

The optically visible iris-pupil complex expansion part may include: a maximum-thickness portion having a maximum thickness between the inner annular edge and the outer annular edge;
an inner area formed relatively close to the opening and disposed between the inner annular edge and the maximum-thickness portion; and
an outer area formed relatively far from the opening and disposed between the maximum-thickness portion and the outer annular edge.

For example, the inner area and the outer area disposed on opposite sides of the maximum-thickness portion may have an asymmetrical shape or an asymmetrical thickness profile.

For example, the inner area may be formed of a relatively soft material so as to adaptively deform to fit the optically visible iris-pupil complex of each implant recipient.

The outer area may be formed of a relatively rigid material so as to maintain a smoothly circular appearance against deformation of the inner area and to reinforce the flexibility of the inner area, thereby providing support rigidity of the ocular implant.

For example, the inner area may be formed with a thinner thickness than the outer area, the inner area extending to an irregular edge between a cornea having a large-curvature portion that convexly protrudes forward and a sclera having a small-curvature portion extending posteriorly from the cornea, among the membrane tissues surrounding the entire eyeball to maintain its shape and adhering closely to the irregular edge so as to prevent the formation of a gap between the irregular edge and the inner area.

For example, the inner area and the outer area may be formed of the same material.

The thickness of the inner area may be formed with a relatively smaller thickness than the outer area.

For example, the optically visible iris-pupil complex expansion part may include an anterior curved surface and a posterior inclined surface that define the thickness of the optically visible iris-pupil complex expansion part.

A first average thickness of the inner area and a second average thickness of the outer area, formed by the anterior curved surface and the posterior inclined surface, may satisfy the relationship that first average thickness<second average thickness.

For example, based on the posterior inclined surface providing a reference for the thickness of the optically visible iris-pupil complex expansion part,
an anterior curved surface forming the inner area may extend along a trajectory relatively close to the posterior inclined surface, and an anterior curved surface forming the outer area may extend along a trajectory relatively farther from the posterior inclined surface, thereby forming a difference in thickness between the inner area and the outer area disposed on both sides of the maximum-thickness portion.

For example, the anterior curved surface forming the inner area may extend along a trajectory relatively close to the posterior inclined surface, forming a relatively thin thickness, and may then form a gently rounded edge at the inner annular edge along a relatively gentle downward curve toward the posterior inclined surface.

The anterior curved surface forming the outer area may extend along a trajectory relatively farther from the posterior inclined surface, forming a relatively greater thickness, and may then form a sharply rounded edge at the outer annular edge along a relatively steep downward curve toward the posterior inclined surface.

For example, the anterior curved surface forming the inner area may follow a downward curve from the maximum-thickness portion toward the inner annular edge, and then form a gently rounded inner annular edge along a relatively steeper downward curve through an inner inflection point, thereby finishing one side of the ocular implant.

The anterior curved surface forming the outer area may follow a downward curve from the maximum-thickness portion toward the outer annular edge, and then form a sharply rounded outer annular edge along a relatively steeper downward curve through an outer inflection point, thereby finishing the other side of the ocular implant.

For example, along the inclination direction of the posterior inclined surface, the distance from the outer annular edge to the outer inflection point may be relatively longer than the distance from the inner annular edge to the inner inflection point.

For example, the inner inflection point and the outer inflection point may respectively correspond to points having the greatest curvature change along profiles of the anterior curved surfaces forming the inner and outer areas.

An ocular implant according to one embodiment of the present invention is permanently implanted on the eyeball of an implant recipient to provide an enlarged appearance of the implant recipient's optically visible iris-pupil complex,
the ocular implant may include:
   a central opening configured to receive light incident on the pupil that provides an optical aperture of the eyeball;
   an inner annular edge surrounding and defining the opening;
   an outer annular edge disposed opposite the inner annular edge that defines the opening and surrounds the opening together with the inner annular edge; and
   an adaptive stretchable part configured to provide adaptive longitudinal stretching along the perimeter of at least one of the inner annular edge and the outer annular edge.

For example, the adaptive stretchable part may provide longitudinal stretching along the perimeter of the inner annular edge so as to adaptively deform to fit the shape and size of the optically visible iris-pupil complex of each implant recipient.

For example, the inner annular edge may be formed in an oval shape having different major and minor axis lengths.

The adaptive stretchable part may provide longitudinal stretching along the major-axis direction and/or the minor-axis direction so as to adaptively form an inner annular edge suitable for the optically visible iris-pupil complex of the implant recipient, between an elongated oval in which the relatively major axis is extended and/or the relatively minor axis is contracted, and a near-circular oval in which the relatively major axis is contracted and/or the relatively minor axis is extended.

For example, the adaptive stretchable part may include at least one slit formed along the perimeter of the inner annular edge.

For example, the adaptive stretchable part may include a plurality of slits formed to be spaced apart from each other along the perimeter of the inner annular edge.

For example, the plurality of slits may be formed at uniform intervals spaced apart from each other along the perimeter of the inner annular edge.

For example, the adaptive stretchable part may include a group of slits formed at opposite positions along a major axis and/or at opposite positions along a minor axis.

For example, the adaptive stretchable part may include:
first and second groups of slits formed at opposite positions along the minor axis, through which divided segments overlap each other to extend the minor axis length while shortening the major axis length; and
third and fourth groups of slits formed at opposite positions along the major axis, through which the divided segments overlap each other to extend the major axis length while shortening the minor axis length.

For example, the slits may be formed at angular positions spaced apart by a constant interval, with the center of the opening, where the major and minor axes of the inner annular edge intersect, serving as a rotation center.

For example, the slits may include:
first and second groups of slits formed at angular positions of 0 degrees and 180 degrees, respectively, corresponding to opposite ends along the minor axis; and
third and fourth groups of slits formed at angular positions of 90 degrees and 270 degrees, respectively, corresponding to opposite ends along the major axis.

For example, the slits may extend into the optically visible iris-pupil complex expansion part along a depth direction from the inner annular edge to the outer annular edge.

For example, the slits may be formed in a radial direction extending inward from the center of the opening, where the major and minor axes intersect, into the optically visible iris-pupil complex expansion part, with the depth direction corresponding to the radial direction.

For example, the adaptive stretchable part may be configured to expand the length of the inner annular edge by allowing the segments divided by the slits along the perimeter of the inner annular edge to overlap each other.

For example, the optically visible iris-pupil complex expansion part may include:
a posterior inclined surface disposed to face the eyeball so as to closely adhere to the eyeball; and
an anterior curved surface disposed toward the external space opposite to the eyeball and forming a thickness profile based on the posterior inclined surface, wherein the anterior curved surface contacts the posterior inclined surface at a position adjacent to the opening to form an inner annular edge, and also contacts the posterior inclined surface at a position opposite the opening to form an outer annular edge, and forms a thickness profile based on the posterior inclined surface to provide a relatively thin inner area and a relatively thick outer area based on a maximum-thickness portion that defines the maximum thickness between the inner annular edge and the outer annular edge; and

In the optically visible iris-pupil complex expansion part, the divided segments formed through the slits constituting the adaptive stretchable part may overlap each other to expand the length of the inner annular edge, thereby forming an additional thickness that increases the thickness of the inner area into which the slit extends from the inner annular edge

For example, the adaptive stretchable part may be formed on the inner annular edge that surrounds the outer ring of the optically visible iris-pupil complex at a position relatively closer to the outer annular edge, so as to adaptively deform to fit the shape and size of each implant recipient's optically visible iris-pupil complex.

To maintain a smoothly circular appearance against deformation of the inner annular edge, the adaptive stretchable part may not be formed on the outer annular edge.

For example, the inner annular edge, in which the segmented pieces overlap each other through the slits forming the adaptive stretchable part, may be formed with a relatively gently rounded edge shape.

The outer annular edge may be formed with a relatively sharply rounded edge shape.

An ocular implant according to one embodiment of the present invention is permanently implanted on the eyeball of an implant recipient to provide an enlarged appearance of the implant recipient's optically visible iris-pupil complex,
the ocular implant may include:
   a central opening configured to receive light incident on the pupil that provides an optical aperture of the eyeball;
   an inner annular edge surrounding and defining the opening;
   an outer annular edge disposed opposite the inner annular edge that defines the opening and surrounds the opening together with the inner annular edge; and
   an optically visible iris-pupil complex expansion part formed between the inner annular edge and the outer annular edge, the optically visible iris-pupil complex expansion part including first and second ends that are separated from each other through an incision and are joined to each other through respective surgical holes formed therein.

For example, in the optically visible iris-pupil complex expansion part between the inner annular edge and the outer annular edge,
an oval shape of the inner annular edge may be formed within the circular shape of the outer annular edge such that positions on opposite sides along the minor axis of the inner annular edge have a relatively wider width than positions on opposite sides along the major axis of the inner annular edge.

The incision, the first and second ends separated from each other through the incision, and the surgical holes formed on the first and second ends may be formed at any one of the positions on opposite sides along the relatively wide minor axis.

For example, in the optically visible iris-pupil complex expansion part between the inner annular edge and the outer annular edge,
the positions on opposite sides along the minor axis of the inner annular edge may be formed with a relatively smaller curvature than the positions on opposite sides along the major axis of the inner annular edge.

The surgical holes may be formed at any one of the positions along the minor axis having a relatively smaller curvature.

For example, the incision may be formed along the minor axis of the inner annular edge.

The first and second ends separated from each other through the incision may be joined to each other along the major axis of the inner annular edge.

Each of the first and second ends may have a plurality of surgical holes arranged along the major-axis direction of the inner annular edge.

For example, the surgical holes may include:
a first surgical hole through which a suture passes to connect the first and second ends to each other; and
a second surgical hole through which an implantation insertion tool passes to pull the ocular implant so that the ocular implant travels along the outer ring of the implant recipient's optically visible iris-pupil complex through an incision in a conjunctiva on the sclera.

For example, the first surgical hole may be formed with a relatively small diameter.

The second surgical hole may be formed with a relatively large diameter.

For example, the first surgical hole may be formed at a position relatively close to the incision.

The second surgical hole may be formed at a position relatively farther from the incision.

An ocular implant according to one embodiment of the present invention may be permanently implanted on the eyeball of an implant recipient to provide an enlarged appearance of the implant recipient's optically visible iris-pupil complex,
the ocular implant may include:
   a central opening configured to receive light incident on the pupil that provides an optical aperture of the eyeball;
   an inner annular edge surrounding and defining the opening;
   an outer annular edge disposed opposite the inner annular edge that defines the opening and surrounds the opening together with the inner annular edge; and
   an optically visible iris-pupil complex expansion part formed between the inner annular edge and the outer annular edge, the optically visible iris-pupil complex expansion part including first and second ends that are separated from each other through an incision and form a catch-engaging or hook-engaging connection with each other.

For example, in the optically visible iris-pupil complex expansion part between the inner annular edge and the outer annular edge,
an oval shape of the inner annular edge may be formed within the circular shape of the outer annular edge such that positions on opposite sides along the minor axis of the inner annular edge have a relatively wider width than positions on opposite sides along the major axis of the inner annular edge.

The incision, the first and second ends separated from each other through the incision, and the catch-engaging or hook-engaging connection structure between the first and second ends may be formed at any one of the positions on opposite sides along the relatively wide minor-axis direction.

For example, the catch-engaging or hook-engaging connection structure may include:
first and second slits formed in the first and second ends, respectively.

For example, at least a portion of the first and second slits may be formed in complementary shapes so as to form a catch-engaging or hook-engaging connection with each other, thereby being fitted together.

For example, the first and second slits may include:
a first portion extending along the major-axis direction of the inner annular edge; and
a second portion extending from the first portion toward the inner annular edge or the outer annular edge along the minor-axis direction of the inner annular edge, or along an oblique direction that follows both the minor and major-axis directions, and opening to the outside of the ocular implant.

For example, the first portions of the first and second slits may extend in parallel along the major-axis direction of the inner annular edge.

The second portions of the first and second slits may be formed in complementary shapes such that the second portion of one of the slits extends toward the outer annular edge, while the second portion of the other slit extends toward the inner annular edge, in opposite directions, and both open to the outside of the ocular implant.

For example, the incision may be formed along the minor-axis direction of the inner annular edge.

The first and second ends may be arranged adjacent to each other along the major-axis direction of the inner annular edge.

Either one of the first and second slits may include an array of multiple slits formed on the first or second end along the major-axis direction, while the other slit may be formed as a single slit.

For example, the catch-engaging or hook-engaging connection structure may include:
an assembly slit formed in the first end; and
an assembly hole formed in the second end, into which the assembly slit is fitted.

For example, an assembly guide unit may be formed on the first end and positioned forward of the assembly slit along an assembly direction between the first and second ends.

For example, the assembly guide unit may include:
a front-end protrusion formed with a relatively narrow width at a front end of the first end along the assembly direction of the first and second ends; and a rear end portion formed with a relatively wider width than a neck portion of the assembly slit, the rear end portion being located rearward of the front-end protrusion and forward of the assembly slit.

The assembly guide unit may further include a variable-width portion that converges toward the protrusion along a diagonal direction following both the major-axis direction corresponding to the assembly direction and the minor-axis direction intersecting the major-axis direction, so as to connect differential widths between the front-end protrusion having a relatively narrow width and the rear end portion having a relatively wide width.

For example, the rear end of the assembly guide unit may be connected to the full width of the first end interposed between the assembly guide unit and the assembly slit.

For example, a tear line including an array of multiple perforations may be formed between the assembly guide unit and the full width of the first end.

For example, the assembly slit may extend inward from both the inner annular edge and the outer annular edge along the minor-axis direction of the inner annular edge to form a neck having a minimum width.

When the neck of the assembly slit is fitted into the assembly hole on the second end side, the relatively wide full width of the first end, formed before and after the assembly slit, may prevent separation between the first and second ends.

### Mode for Carrying Out Invention

Hereinafter, an ocular implant 100 according to a preferred embodiment of the present invention will be described with reference to the accompanying drawings.

FIG. 1 is a schematic view illustrating the structure of an eyeball into which an ocular implant according to one embodiment of the present invention is permanently implanted.

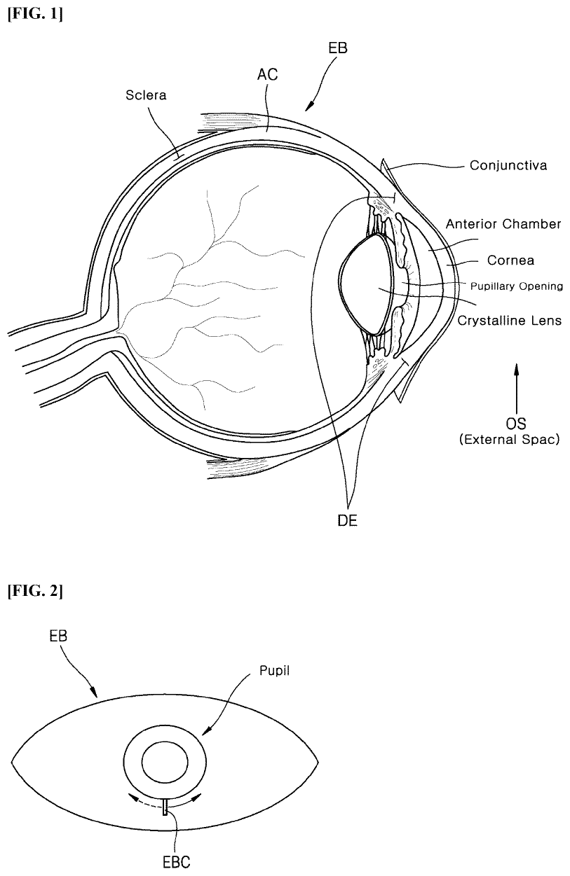

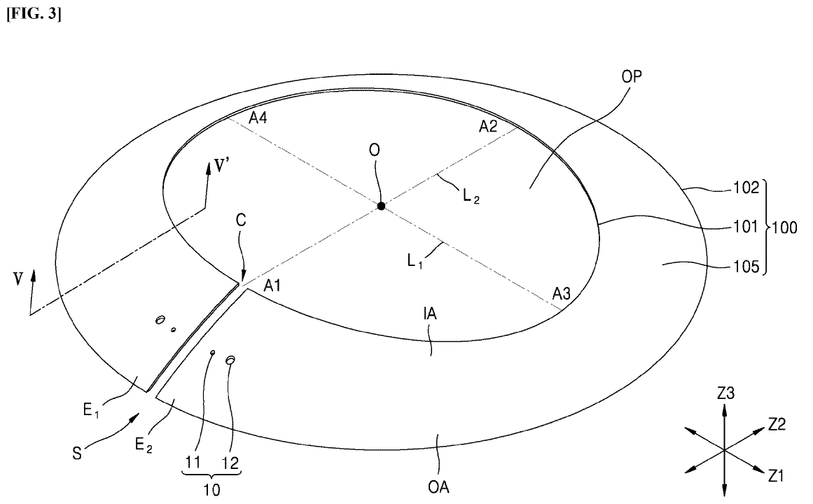

FIG. 2 is a view describing a procedure for implanting an ocular implant according to one embodiment of the present invention, which shows an incision formed at a selected location along an outer ring of the optically visible iris-pupil complex.

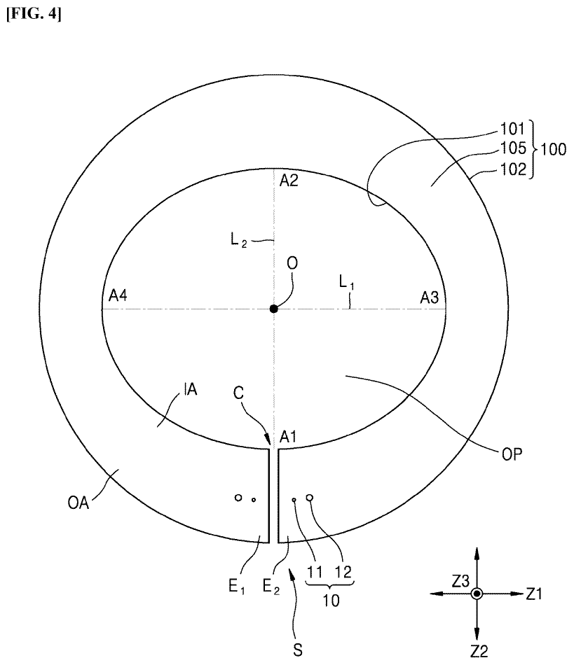

FIG. 3 is a perspective view illustrating an ocular implant according to one embodiment of the present invention.

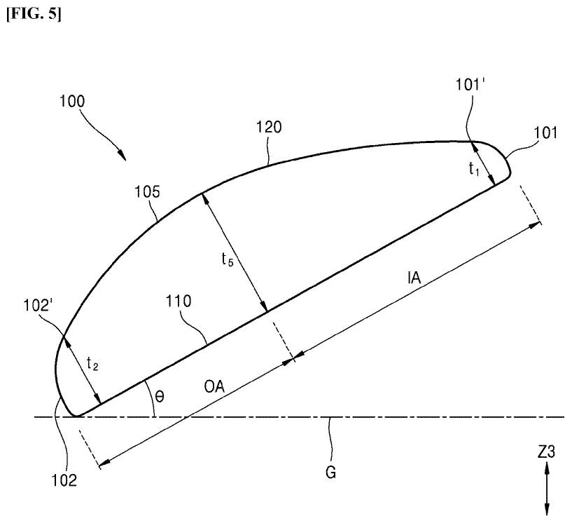

FIG. 4 is a plan view illustrating the ocular implant shown in FIG. 3 as viewed from the front.

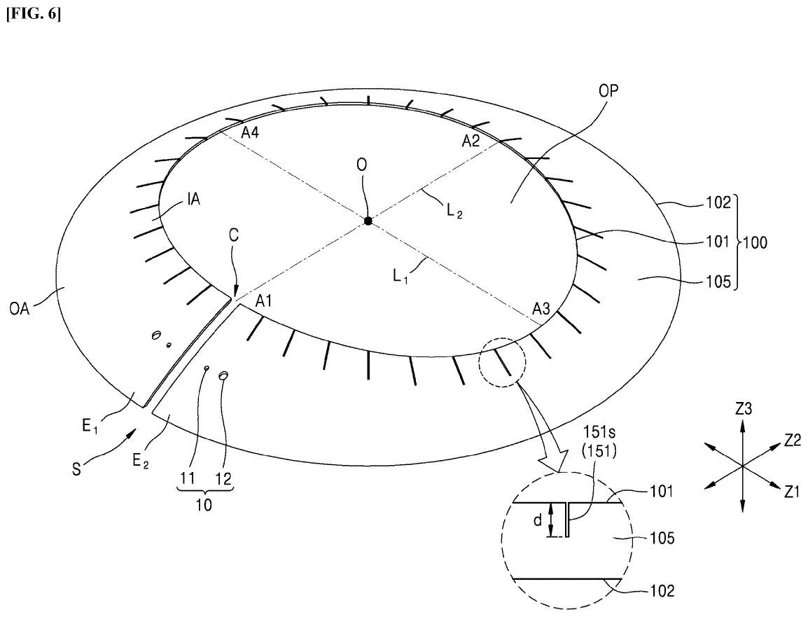

FIG. 5 is a cross-sectional view taken along line V-V′ of FIG. 3.

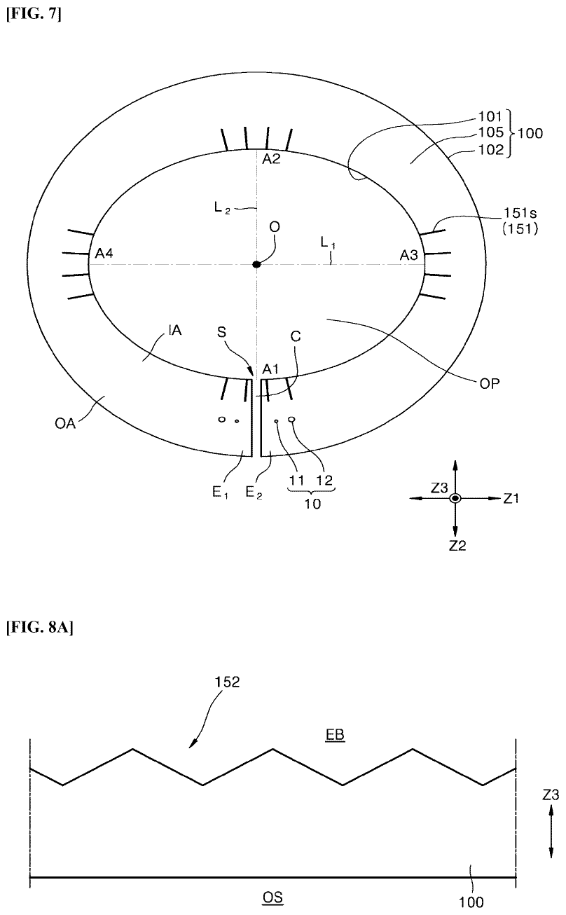

FIG. 6 is a perspective view describing an adaptive stretchable part applied to the ocular implant according to one embodiment of the present invention.

FIG. 7 is a perspective view describing a modified example of the adaptive stretchable part shown in FIG. 6, applied to the ocular implant according to one embodiment of the present invention.

FIGS. 8A and 8B are cross-sectional views of the ocular implant taken along a circumferential direction of the optically visible iris-pupil complex expansion part, which describe different roughened surfaces having isotropic and anisotropic rotational resistance, respectively.

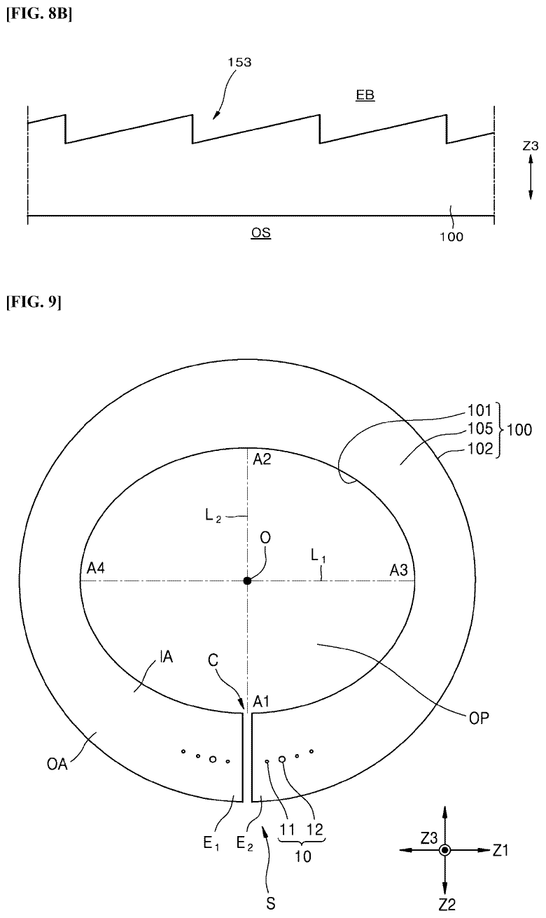

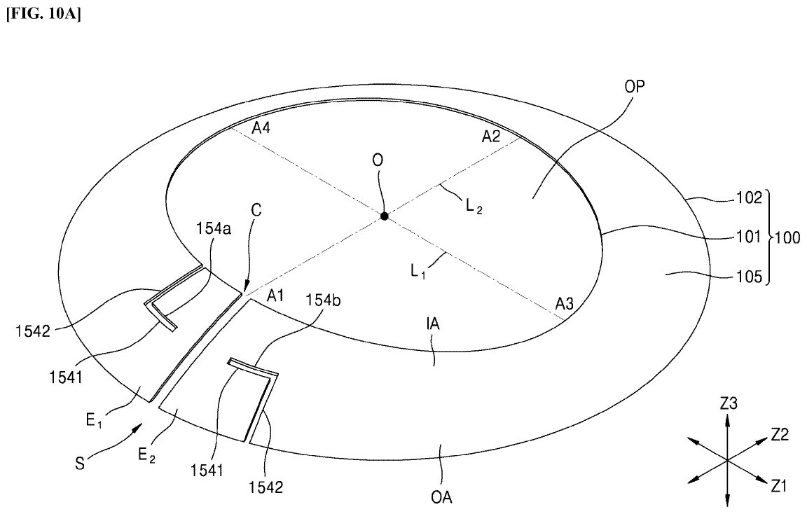

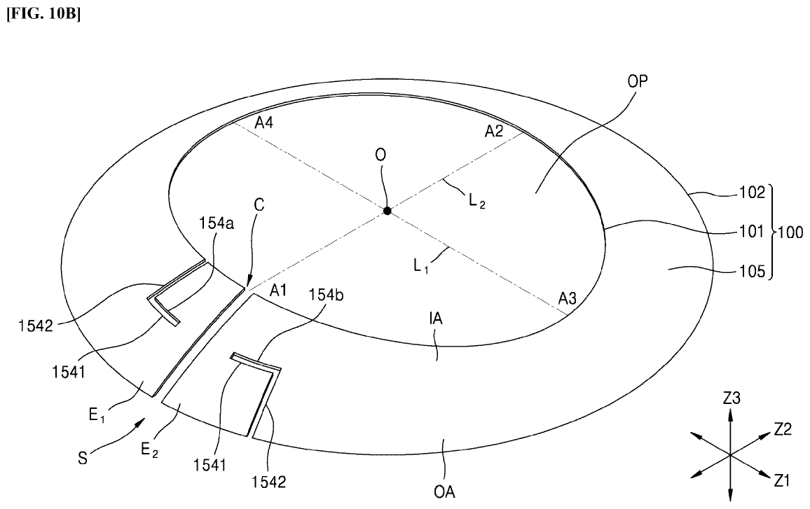

FIG. 9 is a plan view of the ocular implant, taken from a frontal direction, illustrating surgical holes applied to the ocular implant according to one embodiment of the present invention.

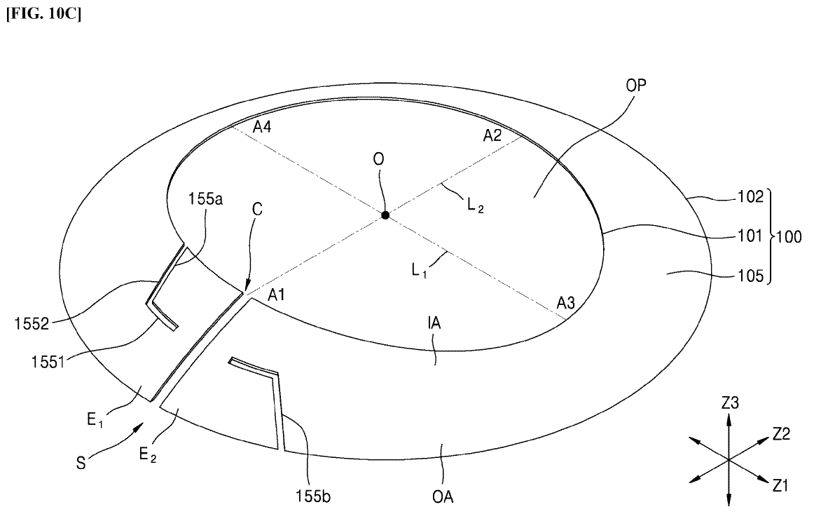

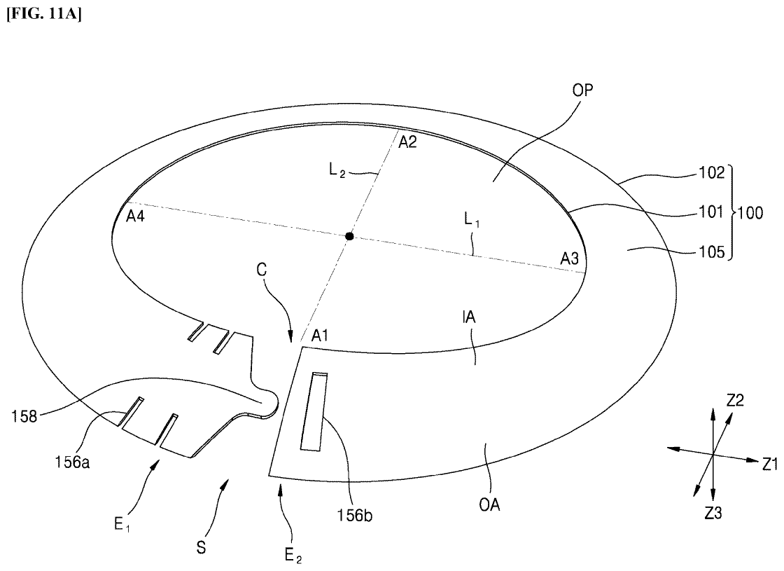

FIGS. 10A to 10C are perspective views illustrating catch-engaging or hook-engaging connections between first and second slits applied to ocular implants according to different embodiments of the present invention.

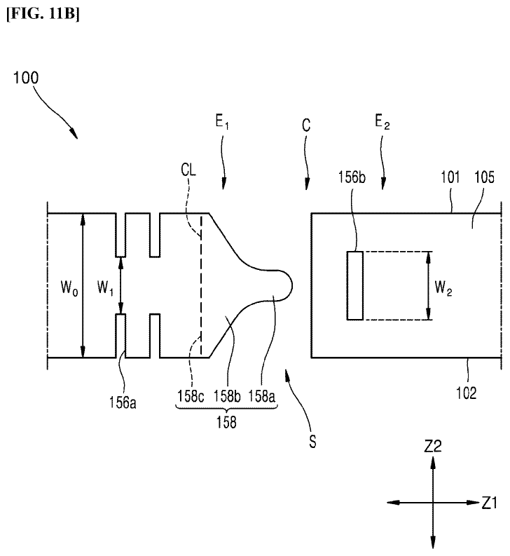

FIGS. 11A and 11B are views illustrating catch-engaging or hook-engaging connections between assembly slits and assembly holes applied to the ocular implant according to one embodiment of the present invention.

Hereinafter, in the ocular implant 100 according to one aspect of the present invention, a optically visible iris-pupil complex expansion part 105 formed with a variable thickness between an inner annular edge 101 and an outer annular edge 102, an anterior curved surface 120 forming a variable-thickness profile on a posterior inclined surface 110 of the optically visible iris-pupil complex expansion part 105, and circular and elliptical profiles formed respectively by the inner annular edge 101 and the outer annular edge 102 will be described.

FIG. 1 is a schematic view illustrating the structure of an eyeball into which an ocular implant according to one embodiment of the present invention is permanently implanted.

FIG. 2 is a view describing a procedure for implanting an ocular implant according to one embodiment of the present invention, which shows an incision formed at a selected location along an outer ring of the optically visible iris-pupil complex.

FIG. 3 is a perspective view illustrating an ocular implant according to one embodiment of the present invention.

FIG. 4 is a plan view illustrating the ocular implant shown in FIG. 3 as viewed from the front.

FIG. 5 is a cross-sectional view taken along line V-V′ of FIG. 3.

The ocular implant 100 according to one embodiment of the present invention is permanently implanted on the implant recipient's eyeball EB to provide the implant recipient's optically visible iris-pupil complex with an enlarged appearance.

The ocular implant 100 may include:
a central opening OP configured to receive light incident on the pupil that provides an optical aperture of an eyeball EB;
the inner annular edge 101 defining the opening OP while surrounding the opening OP;
the outer annular edge 102 disposed opposite the inner annular edge 101 that defines the opening OP and surrounds the opening OP together with the inner annular edge 101; and
the optically visible iris-pupil complex expansion part 105 formed with a variable thickness that changes from the inner annular edge 101 to the outer annular edge 102 between the inner annular edge 101 and the outer annular edge 102.

For example, the inner annular edge 101 and the outer annular edge 102 may surround the opening OP along different contours.

The inner annular edge 101 may be formed in an oval shape along the contour of the optically visible iris-pupil complex so as not to cover the optically visible iris-pupil complex but to provide an appearance integrated with the optically visible iris-pupil complex. The inner annular edge may be shaped to surround the outer rim of the optically visible iris-pupil complex at an adjacent position.

The outer annular edge 102 may be formed in a circular shape to provide a smoothly circular appearance.

For example, the inner annular edge 101 may be formed in an oval shape having different major and minor axis lengths L₁ and L₂.

The major axis length L₁ may be oriented along the direction in which the implant recipient's pair of eyes face each other.

The minor axis length L₂ may be formed oriented along a direction perpendicular to the major axis length L₁.

For example, in the optically visible iris-pupil complex expansion part 105 between the inner annular edge 101 and the outer annular edge 102 may be formed such that one position along the minor-axis direction Z2 of the inner annular edge 101 has a greater width than the opposite position.

To this end, the oval shape of the inner annular edge 101 may be positioned in an offset manner within the circular shape of the outer annular edge 102, biased toward the opposite side.

For example, the optically visible iris-pupil complex expansion part 105 may include:
the posterior inclined surface 110 arranged toward the eyeball EB so as to closely adhere to the eyeball EB; and
the anterior curved surface 120 disposed toward an external space OS opposite to the eyeball EB.

For example, the inner annular edge 101 and the outer annular edge 102 each from the anterior portion of the eyeball EB and appear as an ellipse and a circle when viewed along a frontal direction Z3 from the anterior portion of the eyeball EB, where a crystalline lens that performs a lens function and a pupil forming a light entrance aperture are located, toward a posterior portion of the eyeball EB.

The posterior inclined surface 110 may be formed at a constant tilting angle θ from a vertical plane G perpendicular to the frontal direction Z3, such that the inner annular edge 101 and the outer annular edge 102 define an anterior position and a posterior position, respectively.

For example, the posterior inclined surface 110 may provide a support surface that is supported on an inclined surface formed in a packaging container of the ocular implant 100.

For example, the anterior curved surface 120 may be formed along a spline curve whose curvature or radius of curvature varies along the inclination direction of the posterior inclined surface 110.

For example, the optically visible iris-pupil complex expansion part 105 may have a variable thickness such that the thickness between the posterior inclined surface 110 and the anterior curved surface 120 varies. In this case, the thickness of the optically visible iris-pupil complex expansion part 105 may be measured along a direction perpendicular to the posterior inclined surface 110, from the posterior inclined surface 110 toward the anterior curved surface 120.

For example, the inner annular edge 101 may be formed by contact between the anterior curved surface 120 and the posterior inclined surface 110, which define the thickness of the optically visible iris-pupil complex expansion part 105, at a position adjacent to the opening OP.

For example, the outer annular edge 102 may be formed by contact between the anterior curved surface 120 and the posterior inclined surface 110, which define the thickness of the optically visible iris-pupil complex expansion part 105, at a position opposite to the inner annular edge 101 that defines the opening OP.

For example, the anterior curved surface 120 and the posterior inclined surface 110, which define the thickness of the optically visible iris-pupil complex expansion part 105, may come into mutual contact to form the inner annular edge 101 and the outer annular edge 102, each having a rounded edge shape. In this case, the inner annular edge 101 may be formed with a relatively gently rounded edge shape, while the outer annular edge 102 may be formed with a relatively sharply rounded edge shape.

For example, the thickness at the edge of the inner annular edge 101 may be smaller than the thickness at the edge of the outer annular edge 102.

For example, the inner annular edge 101 may extend to an irregular edge DE between a cornea having a large-curvature portion that convexly protrudes forward and a sclera having a small-curvature portion extending posteriorly from the cornea, among the membrane tissues surrounding the entire eyeball EB to maintain its shape, and the inner annular edge 101 may have a relatively gently rounded edge shape that defines one side of the ocular implant 100.

The outer annular edge 102, which closely adheres to the sclera in the large-curvature portion, may have a relatively sharply rounded edge shape that defines the opposite side of the ocular implant 100, so as to prevent lifting from the sclera or the formation of a gap between the sclera and the outer annular edge 102.

For example, the optically visible iris-pupil complex expansion part 105 may include:
a maximum-thickness portion t5 having a maximum thickness between the inner annular edge 101 and the outer annular edge 102;
an inner area IA formed relatively close to the opening OP and disposed between the inner annular edge 101 and the maximum-thickness portion t5; and
an outer area OA formed relatively far from the opening OP and disposed between the maximum-thickness portion t₅ and the outer annular edge 102.

For example, the inner area IA and the outer area OA disposed on both sides of the maximum-thickness portion t₅ may have an asymmetrical shape or an asymmetrical thickness profile.

For example, the inner area IA may be formed of a relatively soft material so as to adaptively deform to fit the optically visible iris-pupil complex of each implant recipient.

The outer area OA may be formed of a relatively rigid material so as to maintain a smoothly circular appearance against deformation in the inner area IA, and to reinforce the flexibility of the inner area IA, thereby providing structural rigidity of the ocular implant 100.

For example, the inner area IA may be formed with a thinner thickness than the outer area OA, the inner area IA extending to an irregular edge DE between a cornea having a large-curvature portion that convexly protrudes forward and a sclera having a small-curvature portion extending posteriorly from the cornea, among the membrane tissues surrounding the entire eyeball EB to maintain its shape and adhering closely to the irregular edge DE so as to prevent the formation of a gap between the irregular edge DE and the inner area IA.

For example, the inner area IA and the outer area OA may be formed of the same material.

the thickness of the inner area IA may be formed with a relatively smaller thickness than the outer area OA.

For example, a first average thickness of the inner area IA and a second average thickness of the outer area OA, formed by the anterior curved surface 120 and the posterior inclined surface 110, may satisfy the relationship first average thickness<second average thickness.

For example, based on the posterior inclined surface 110 providing a reference for the thickness of the optically visible iris-pupil complex expansion part 105,
the anterior curved surface 120 forming the inner area IA may extend along a trajectory relatively close to the posterior inclined surface 110, and the anterior curved surface 120 forming the outer area OA may extend along a trajectory relatively farther from the posterior inclined surface 110, thereby forming a difference in thickness between the inner area IA and the outer area OA disposed on both sides of the maximum-thickness portion t₅.

For example, the anterior curved surface 120 forming the inner area IA may extend along a trajectory relatively close to the posterior inclined surface 110, forming a relatively thin thickness, and may form a gently rounded edge at the inner annular edge 101 along a relatively gentle downward curve toward the posterior inclined surface 110.

The anterior curved surface 120 forming the outer area OA may extend along a trajectory relatively farther from the posterior inclined surface 110, forming a relatively greater thickness, and may then form a sharply rounded edge at the outer annular edge 102 along a relatively steep downward curve toward the posterior inclined surface 110.

For example, the anterior curved surface 120 forming the inner area IA may follow a downward curve from the maximum-thickness portion t5 toward the inner annular edge 101, and may then form a gently rounded inner annular edge 101 along a relatively steeper downward curve through an inner inflection point 101′, thereby finishing one side of the ocular implant 100.

The anterior curved surface 120 forming the outer area OA may follow a downward curve from the maximum-thickness portion t5 toward the outer annular edge 102, and may then form a sharply rounded outer annular edge 102 along a relatively steeper downward curve through an outer inflection point 102′, thereby finishing the other side of the ocular implant 100.

For example, along the inclination direction of the posterior inclined surface 110, the distance from the outer annular edge 102 to the outer inflection point 102′ may be relatively longer than the distance from the inner annular edge 101 to the inner inflection point 101′. In this case, the inner inflection point 101′ and the outer inflection point 102′ may respectively correspond to points having the greatest curvature change along profiles of the anterior curved surfaces 120 forming the inner area IA and the outer area OA.

The ocular implant 100 of the present invention may be provided as a generally ring-shaped member having the central opening OP, through which light can be incident toward the crystalline lens that performs the lens function of the eyeball EB or toward the pupil that defines the optical aperture of the eyeball EB.

The ocular implant 100 may be formed as a ring-shaped member that surrounds the outer ring of the optically visible iris-pupil complex of the implant recipient to enlarge the apparent size of the optically visible iris-pupil complex, and that has the central opening OP allowing light to enter the pupil while surrounding the outer ring of the optically visible iris-pupil complex of the implant recipient.

In one embodiment of the present invention, the ocular implant 100 may include the inner annular edge 101 that surrounds the outer ring of the optically visible iris-pupil complex of the implant recipient; the outer annular edge 102 that, opposite to the inner annular edge 101, surrounds the optically visible iris-pupil complex outer ring together with the inner annular edge 101; and the optically visible iris-pupil complex expansion part 105 formed with a variable thickness that changes from the inner annular edge 101 to the outer annular edge 102 between the inner annular edge 101 and the outer annular edge 102. The optically visible iris-pupil complex expansion part 105 may be defined between the inner annular edge 101 and the outer annular edge 102, which together surround the optically visible iris-pupil complex outer ring at the inner and outer positions, respectively, and may provide an optically visible iris-pupil complex expansion area in the ocular implant 100 according to one embodiment of the present invention. In one embodiment of the present invention, the inner annular edge 101 and the outer annular edge 102 may define the shape of the optically visible iris-pupil complex expansion part 105 constituting the ocular implant 100 or the body of the ocular implant 100, and may define at least a portion of the overall shape of the ocular implant 100.

The ocular implant 100 according to one embodiment of the present invention may be formed into a three-dimensional shape so as to closely adhere to the approximately spherical eyeball EB. A principal function of the ocular implant 100 according to one embodiment of the present invention is to aesthetically enhance the shape of the optically visible iris-pupil complex as viewed from the frontal direction Z3 of the implant recipient's face. In this respect, the shape of the optically visible iris-pupil complex observed from the frontal direction Z3 may be defined by the inner annular edge 101 and the outer annular edge 102. From this viewpoint, hereinafter, the “shapes of the inner annular edge 101 and the outer annular edge 102” may refer to the two-dimensional line profile observed from the frontal direction Z3 of the implant recipient's face. However, the ocular implant 100 according to one embodiment of the present invention is permanently implanted on the eyeball EB of the implant recipient. Due to this reason, the ocular implant 100 may be formed with an appropriate three-dimensional configuration to fit the approximately spherical eyeball EB. For example, the cross-section of the optically visible iris-pupil complex expansion part 105, taken along the frontal direction Z3, may be formed with a variable thickness that changes from the inner expansion edge toward the outer expansion edge.

More specifically, the optically visible iris-pupil complex expansion part 105 may be formed in a three-dimensional configuration so as to closely adhere to the approximately spherical eyeball EB. In a cross-section taken along the frontal direction Z3, the optically visible iris-pupil complex expansion part 105 may include the posterior inclined surface 110 inclined at a constant tilting angle θ from the vertical plane G perpendicular to the frontal direction Z3 so as to fit intimately against the surface of the eyeball EB, and the anterior curved surface 120 disposed toward the external space OS opposite to the eyeball EB.

In one embodiment of the present invention, the eyeball EB is protected by a strong and tough membrane tissue that entirely encloses the eyeball EB, thereby maintaining the overall shape of the eyeball EB. The membrane tissue may include a cornea located on the anterior side through which light is incident, and a sclera that extends posteriorly from the cornea. For example, the cornea performs a lens function to focus incident light onto the retina located at the posterior portion of the eyeball EB, and the cornea may be formed in a position anterior to the optically visible iris-pupil complex, which defines a light-incident opening formed in front of the crystalline lens, and in front of the anterior chamber disposed anterior to the optically visible iris-pupil complex. The cornea may form a large-curvature portion of the membrane tissue having relatively large curvature, while enclosing the anterior portion of the eyeball EB that includes the crystalline lens, the optically visible iris-pupil complex, and the anterior chamber. The sclera may extend posteriorly from the cornea and form a small-curvature portion of the membrane tissue having relatively small curvature. The cornea and the sclera may respectively form the large-and small-curvature portions that surround the eyeball EB and maintain its overall shape, and a geometric discontinuity such as an irregular edge DE may be formed at the boundary between the two portions.

As described above, the cornea and the sclera may respectively form the large- and small-curvature portions of the membrane tissue that entirely surround the eyeball EB and maintain its overall shape. Substantially the same membrane tissue may be formed in a convex shape protruding toward the external space OS opposite to the eyeball EB, while enclosing anterior structures such as the crystalline lens located at the anterior position. In addition, the sclera extending posteriorly from the cornea at the anterior position may have a reduced curvature compared to the cornea. From the scleral region located beyond the irregular edge DE between the cornea and the sclera to a curved portion AC of the sclera, the surface may be formed in a shape closer to an inclined surface rather than a substantially curved contour. The ocular implant 100 according to one embodiment of the present invention may be implanted on the sclera. For example, the ocular implant 100 may be implanted on the scleral region positioned beyond the cornea through which light is incident, that is, on a portion of the sclera located posterior to the corneal region that covers the anterior side of the crystalline lens. In one embodiment of the present invention, the ocular implant 100 may be advanced anteriorly up to the irregular edge DE between the sclera and the cornea. At this time, the cornea located at the anterior position is formed with a relatively large curvature in the region anterior to the crystalline lens, whereas the sclera extending posteriorly from the cornea is formed with a relatively small curvature, thereby defining the irregular edge DE between the cornea and the sclera. In the ocular implant 100 that can extend up to the irregular edge DE, the inner area IA in contact with the opening OP may be formed thinner than the outer area OA positioned farther from the opening OP, so as to accommodate the irregular edge DE between the cornea and the sclera and prevent the formation of a gap between the tissue of the eyeball EB and the implanted ocular implant 100.

In one embodiment of the present invention, the optically visible iris-pupil complex expansion part 105 may include the posterior inclined surface 110 arranged toward the eyeball EB so as to closely contact the eyeball EB, and the anterior curved surface 120 arranged toward the external space OS opposite to the eyeball EB, thereby facing the posterior inclined surface 110. The optically visible iris-pupil complex expansion part 105 may be formed with a variable thickness that changes between the posterior inclined surface 110 and the anterior curved surface 120. In one embodiment of the present invention, the thickness of the optically visible iris-pupil complex expansion part 105 may correspond to a dimension measured in a direction perpendicular to the posterior inclined surface 110 of the posterior inclined surface 110 and the anterior curved surface 120 defining respective portions of the optically visible iris-pupil complex expansion part 105.

In another embodiment of the present invention, the optically visible iris-pupil complex expansion part 105 may be formed with a variable thickness that changes progressively from the inner annular edge 101 to the outer annular edge 102. More specifically, based on the maximum-thickness portion t5 having the maximum thickness between the inner annular edge 101 and the outer annular edge 102, the thickness may gradually decrease toward both sides of the inner annular edge 101 and the outer annular edge 102, such that both ends of the optically visible iris-pupil complex expansion part 105 are smoothly terminated at the inner annular edge 101 and the outer annular edge 102.

In one embodiment of the present invention, the thickness of the optically visible iris-pupil complex expansion part 105 may be defined along a direction perpendicular to the posterior inclined surface 110 between the posterior inclined surface 110 and the anterior curved surface 120. The posterior inclined surface 110 and the anterior curved surface 120 may contact each other at the inner annular edge 101, which is relatively close to the opening OP, and at the outer annular edge 102, which is relatively farther from the opening OP, thereby forming rounded edges at the respective inner annular edge 101 and outer annular edge 102. In one embodiment of the present invention, with reference to the posterior inclined surface 110, the anterior curved surface 120 may define the thickness profile of the optically visible iris-pupil complex expansion part 105 along the posterior inclined surface 110 and may form a trajectory farthest from the posterior inclined surface 110 to provide the maximum-thickness portion t5 between the inner annular edge 101 and the outer annular edge 102. In addition, the anterior curved surface 120 may gradually form a downward trajectory toward the posterior inclined surface 110 on both sides of the maximum-thickness portion t5, thereby forming the inner annular edge 101 and the outer annular edge 102 at opposite sides of the maximum-thickness portion t5. As the anterior curved surface 120 approaches and contacts the posterior inclined surface 110, gently rounded and sharply rounded edges may be formed at the inner annular edge 101 and the outer annular edge 102, respectively.

In one embodiment of the present invention, the maximum-thickness portion t5 of the optically visible iris-pupil complex expansion part 105 may be positioned at a central region between the inner annular edge 101 and the outer annular edge 102. The optically visible iris-pupil complex expansion part 105 may include the inner area IA between the maximum-thickness portion t5 and the inner annular edge 101, that is, the inner area IA relatively adjacent to the central opening OP, and the outer area OA between the maximum-thickness portion t5 and the outer annular edge 102, that is, the outer area OA relatively farther from the central opening OP and positioned opposite to the inner area IA.

In this manner, in one embodiment of the present invention, the optically visible iris-pupil complex expansion part 105 may include the inner area IA positioned relatively close to the opening OP and the outer area OA positioned relatively far from the opening OP, and the optically visible iris-pupil complex expansion part 105 may include the inner area IA and the outer area OA having mutually different thickness deviations. In one embodiment of the present invention, the fact that the inner area IA and the outer area OA of the optically visible iris-pupil complex expansion part 105 have different thickness deviations means that the optically visible iris-pupil complex expansion part 105 including the inner area IA and the outer area OA has an asymmetrical thickness profile or an asymmetrical shape with respect to the maximum-thickness portion t₅ that defines a boundary between the inner area IA and the outer area OA. For example, the anterior curved surface 120 forming the inner area IA may follow a trajectory relatively close to the posterior inclined surface 110, and the anterior curved surface 120 forming the outer area OA may follow a trajectory relatively farther from the posterior inclined surface 110, thereby forming a thickness difference between the inner area IA and the outer area OA positioned on opposite sides of the maximum-thickness portion t₅. In one embodiment of the present invention, the fact that the inner area IA and the outer area OA of the optically visible iris-pupil complex expansion part 105 have different thickness deviations means that, between a thickness integral value of the inner area IA obtained by integrating the thickness between the posterior inclined surface 110 and the anterior curved surface 120 along the inclined-surface direction over the entire length of the inner area IA, and a thickness integral value of the outer area OA obtained by integrating the thickness between the posterior inclined surface 110 and the anterior curved surface 120 along the inclined-surface direction over the entire length of the outer area OA, the thickness integral value of the outer area OA is relatively greater than that of the inner area IA. For example, even when the lengths of the inner area IA and the outer area OA differ along the inclined-surface direction, when comparing a unit-length-based thickness integral value of the inner area IA, which is obtained by dividing the thickness integral value of the inner area IA by the length of the inner area IA along the inclined-surface direction, with a unit-length-based thickness integral value of the outer area OA, which is obtained by dividing the thickness integral value of the outer area OA by the length of the outer area OA along the inclined-surface direction, the thickness integral value per unit length of the outer area OA may be relatively greater than that of the inner area IA. Throughout the present specification, the terms “average thickness of the inner area IA” and “average thickness of the outer area OA” respectively refer to the thickness integral value per unit length of the inner area IA and the thickness integral value per unit length of the outer area OA.

In this manner, in one embodiment of the present invention, the optically visible iris-pupil complex expansion part 105 may form the maximum-thickness portion t₅ at a central region between the inner annular edge 101 and the outer annular edge 102, while forming the inner area IA and the outer area OA having differential average thicknesses on both sides of the maximum-thickness portion t₅. At this time, the inner area IA, which is formed with a relatively smaller average thickness, may extend toward the irregular edge DE defined between the cornea of a large-curvature portion that convexly protrudes forward to include a crystalline lens or the like within the eyeball EB, and the sclera of a small-curvature portion extending rearward from the cornea. By forming the average thickness of the inner area IA relatively small, the inner area IA can be inserted to a sufficient depth up to the irregular edge DE, ensuring close adhesion to the irregular edge DE without forming any gap between the irregular edge DE (between the cornea and the sclera of the eyeball EB) and the inner area IA. To this end, it is preferable that the inner area IA be formed with a relatively small average thickness. For example, in one embodiment of the present invention, the inner area IA may be implanted in a form that is partially inserted into the eyeball EB. The inner area IA may be inserted between the sclera and the conjunctiva through an incision EBC formed in the conjunctiva covering the sclera, and may extend forward to reach the irregular edge DE between the sclera and the cornea of the eyeball EB. Because the inner area IA has a relatively smaller average thickness than the outer area OA, it can be inserted into a narrow implantation space between the sclera and the conjunctiva formed during surgery and can reach the irregular edge DE to a sufficient depth up to the irregular edge DE. If the inner area IA of the optically visible iris-pupil complex expansion part 105 is not formed to be sufficiently thin, there is a possibility that a gap may be created between the inner area IA and the irregular edge DE between the sclera and the cornea. Bacteria may penetrate through such a gap between the inner area IA and the irregular edge DE, which are not in sufficiently close contact, thereby causing side effects such as inflammation (for example, corneal ulcer resulting from infection by bacteria, viruses, or fungi). Furthermore, if the irregular edge DE between the sclera and the cornea and the inner area IA of the optically visible iris-pupil complex expansion part 105 are not tightly conforming to each other, the shape of the optically visible iris-pupil complex observed from the frontal direction Z3 of the implant recipient's face may be visually distorted or collapsed, thereby failing to provide an aesthetically pleasing appearance (causing cosmetic issues). In addition, if the inner area IA of the optically visible iris-pupil complex expansion part 105 is not sufficiently thin, tears may not adequately wet the cornea, which may lead to corneal ulceration due to infection.

In one embodiment of the present invention, the outer area OA may be formed with a relatively greater average thickness compared to the inner area IA. The outer area OA, having such a relatively high average thickness, may provide overall support rigidity to the ocular implant 100. For example, in one embodiment of the present invention, the ocular implant 100 may be formed of a flexible material such as silicone, which is biocompatible and capable of flexibly conforming to the surface of the eyeball EB. Even when formed of a flexible material, a certain degree of support rigidity should preferably be ensured in consideration of handling convenience during production, packaging, and distribution, as well as ease of surgical implantation. To this end, unlike the inner area IA having a relatively low average thickness, advantageous for adhering closely to the surface or a part of the tissue of the eyeball EB, even in structural regions disadvantageous for adhesion such as the irregular edge DE, the outer area OA may be formed with a relatively high average thickness in consideration of the overall support rigidity of the ocular implant 100. As described below, in one embodiment of the present invention, since the ocular implant 100 is permanently implanted on the eyeball EB, it may be made of a flexible material having excellent adaptability to the eyeball EB. However, even when formed from a single material, the ocular implant 100 may include the inner area IA exhibiting relatively high flexibility and the outer area OA exhibiting relatively high rigidity, which are achieved through differences in shape and thickness, thereby maintaining sufficient structural support while ensuring proper conformity to the eyeball EB.

In one embodiment of the present invention, the inner area IA may be formed of a relatively soft material so as to adaptively deform to the optically visible iris-pupil complex shape of each implant recipient. The outer area OA may be formed of a relatively rigid material so as to maintain a circular external appearance against deformation in the inner area IA and to reinforce the flexibility of the inner area IA, thereby providing support rigidity to the ocular implant 100.

In one embodiment of the present invention, the inner area IA needs to be closely fitted to a geometric singularity such as the irregular edge DE formed between the sclera and the cornea. To this end, for example, the inner area IA may provide relatively excellent flexibility and may be formed with a relatively low average thickness, so that it can be finely adjusted according to the size of the cornea and adhere as closely as possible to the corneal region adjacent to the sclera. Meanwhile, the outer area OA may be positioned on the sclera at a location relatively distant from the geometric singularity such as the irregular edge DE between the sclera and the cornea, and may be placed on a region of the sclera having a relatively small curvature. For example, the outer area OA may be positioned on a portion of the sclera beyond the irregular edge DE between the cornea and the sclera, on a region of the sclera having a curvature closer to an inclined surface than to the curved surface extending from the scleral region beyond the irregular edge DE to the curved portion AC of the sclera, where flexibility for adhesion to the sclera is less required. Particularly, in order to supplement the rigidity of the inner area IA having a relatively low average thickness and to provide overall supporting rigidity to the ocular implant 100, the outer area OA may be formed with a relatively high average thickness. For example, in one embodiment of the present invention, the first average thickness of the inner area IA and the second average thickness of the outer area OA, which are defined by the posterior inclined surface 110 and the anterior curved surface 120, may satisfy the relationship that first average thickness<second average thickness.

In one embodiment of the present invention, the opening OP may be formed in an open configuration so as not to interfere with the incidence of light, for example, so as not to obstruct the light entering the pupil that provides the optical aperture of the eyeball EB. As a result, the flexibility of the inner area IA of the optically visible iris-pupil complex expansion part 105 can be enhanced through the opening OP. For example, in one embodiment of the present invention, the inner area IA is preferably positioned to be integrally observed with the optically visible iris-pupil complex that forms the light-receiving optical aperture of the eyeball EB, while surrounding the outer ring of the optically visible iris-pupil complex adjacent thereto, without covering or obscuring the optically visible iris-pupil complex itself. In particular, considering that the shape and size of the optically visible iris-pupil complex may differ depending on the physical conditions of each implant recipient, it is preferable that the inner area IA have an adaptively extendable length capable of providing sufficient flexibility to surround the outer ring of each different optically visible iris-pupil complex. To this end, unlike conventional techniques in which the central portion is covered with a transparent material to prevent light obstruction, the embodiment of the present invention allows light to pass freely through the central opening OP while providing sufficient flexibility to the inner area IA via the boundary of the opening OP. This enables permanent implantation customized to the physical condition of each implant recipient, such as the shape and size of the optically visible iris-pupil complex, without interfering with the passage of light. As described below, in one embodiment of the present invention, the inner area IA may include an adaptive stretchable part 151 capable of providing longitudinal extension adaptively so as to surround optically visible iris-pupil complex of various shapes and sizes at the outer ring region most adjacent to the optically visible iris-pupil complex, even under individual anatomical variations among implant recipients. The adaptive stretchable part 151 may be formed at a position adjacent to the inner annular edge 101 that defines the inner area IA of the optically visible iris-pupil complex expansion part 105, for example, the end of the inner area IA therein. However, in various embodiments of the present invention, even without an additional adaptive stretchable part 151, a certain degree of longitudinal stretching may still be achieved through the inherent flexibility of the inner area IA itself, which is formed with a relatively low average thickness. Accordingly, the inner area IA can undergo deformation customized to the shape and size of each individual optically visible iris-pupil complex while maintaining optical transparency and anatomical conformity.

In one embodiment of the present invention, the optically visible iris-pupil complex expansion part 105 may include the inner area IA having a relatively low average thickness and the outer area OA having a relatively high average thickness. The optically visible iris-pupil complex expansion part 105 may therefore have an asymmetrical shape defined by the inner area IA and the outer area OA on opposite sides having different average thicknesses with respect to the maximum-thickness portion t5 formed at a central position between the inner annular edge 101 and the outer annular edge 102. In one embodiment of the present invention, the inner area IA and the outer area OA may have an asymmetrical configuration based on the maximum-thickness portion t5, and the inner annular edge 101 and the outer annular edge 102, which respectively define the ends of the inner area IA and the outer area OA, may also have asymmetrical shapes relative to each other.

In one embodiment of the present invention, the optically visible iris-pupil complex expansion part 105 may include the posterior inclined surface 110 arranged toward the eyeball EB so as to closely adhere to the eyeball EB, and the anterior curved surface 120 arranged toward the external space OS opposite to the eyeball EB and forming a thickness profile with respect to the posterior inclined surface 110. The anterior curved surface 120 may form the inner annular edge 101 by contacting the posterior inclined surface 110 at a position adjacent to the opening OP, and the outer annular edge 102 by contacting the posterior inclined surface 110 at a position opposite the opening OP. Based on the maximum-thickness portion t5 having the maximum thickness between the inner annular edge 101 and the outer annular edge 102, a thickness profile may be formed with respect to the posterior inclined surface 110 so as to define a relatively thin inner area IA and a relatively thick outer area OA.

In one embodiment of the present invention, with reference to the posterior inclined surface 110, the anterior curved surface 120 that defines a thickness profile on the posterior inclined surface 110 may form a profile having variations in height and depth in the thickness direction along the inclined surface direction of the posterior inclined surface 110, thereby forming a variable thickness from the posterior inclined surface 110. For example, the anterior curved surface 120 forming such a thickness profile may be formed along a spline curve in which the curvature or radius of curvature varies along the inclined surface direction of the posterior inclined surface 110.

In one embodiment of the present invention, the inner annular edge 101 and the outer annular edge 102 may each be formed in a rounded edge shape where the anterior curved surface 120 and the posterior inclined surface 110 that define the thickness of the optically visible iris-pupil complex expansion part 105 meet each other. That is, in one embodiment of the present invention, both the inner annular edge 101 and the outer annular edge 102 may be formed in rounded edge shapes rather than in angular corner shapes. Even though both the inner annular edge 101 and the outer annular edge 102 are formed in rounded edge shapes, the curvature formed at the corner of the inner annular edge 101 and the curvature formed at the corner of the outer annular edge 102 may be differentially defined. For example, in one embodiment of the present invention, the curvature at the corner of the inner annular edge 101 may be smaller than that at the corner of the outer annular edge 102. In other words, the radius of curvature of the inner annular edge 101 may be larger than that of the outer annular edge 102.

In one embodiment of the present invention, the fact that the curvature of the edge of the inner annular edge 101 is set smaller than the curvature of the edge of the outer annular edge 102 may mean that the edge of the inner annular edge 101 is formed with a relatively more rounded shape, that is, formed with a gentler curvature. In other words, it may mean that the edge of the outer annular edge 102 is formed with a relatively sharply rounded shape.

In one embodiment of the present invention, the inner annular edge 101 may extend to an irregular edge between a cornea having a large-curvature portion that convexly protrudes forward and a sclera having a small-curvature portion extending posteriorly from the cornea, among the membrane tissues surrounding the entire eyeball EB to maintain its shape, and the inner annular edge 101 may have a relatively gently rounded edge shape that defines one side of the ocular implant 100. The outer annular edge 102, which closely adheres to the sclera in the large-curvature portion, may have a relatively sharply rounded edge shape that defines the opposite side of the ocular implant 100, so as to prevent lifting from the sclera or the formation of a gap between the sclera and the outer annular edge 102.

In one embodiment of the present invention, the inner area IA needs to be closely adhered to a geometric singularity such as the irregular edge DE formed by the sclera and the cornea. For example, the inner area IA may be formed with a relatively low average thickness so as to be finely adjusted according to the size of the cornea and to ensure the closest possible contact with the corneal surface.

Accordingly, the inner area IA may be formed with a relatively low average thickness in consideration of adhesion to the irregular edge DE of the eyeball EB and may be finished with a relatively gently rounded inner annular edge 101 in view of minimizing potential damage to the internal tissue of the eyeball EB. In one embodiment of the present invention, the inner annular edge 101, which is inserted into the interior of the eyeball EB or into a relatively complex implantation space, such as a narrow implantation space or the irregular edge DE, and which comes into contact with the internal tissue of the eyeball EB, may be formed with a relatively gently rounded shape, thereby preventing irritation or damage to the internal tissue of the eyeball EB during the permanent implantation of the ocular implant 100.

In one embodiment of the present invention, the inner annular edge 101 can prevent irritation or damage to the internal tissue of the eyeball EB through its relatively gently rounded shape. Meanwhile, the outer annular edge 102 can block adverse effects such as the penetration of foreign substances or bacteria through its relatively sharply rounded shape. The ocular implant 100 according to one embodiment of the present invention may be implanted on the scleral surface in a state that is relatively lifted from the surface of the sclera through the rounded edge of the outer annular edge 102. For example, a minute clearance between the ocular implant 100 and the sclera may be provided through the rounded edge of the outer annular edge 102. In one embodiment of the present invention, the edge of the outer annular edge 102 is formed relatively sharply. That is, the anterior curved surface 120 forming the thickness of the outer area OA may define a relatively distant trajectory from the posterior inclined surface 110 to form a relatively high average thickness, and then form a sharply rounded edge at the outer annular edge 102 along a relatively steep downward curve. In this manner, as the anterior curved surface 120 forming the thickness of the outer area OA approaches the outer annular edge 102, it follows a steep downward curve to form a sharply rounded outer annular edge 102 together with the posterior inclined surface 110. By finishing the optically visible iris-pupil complex expansion part 105 through this sharply rounded outer annular edge 102, side effects such as infection or inflammation due to the penetration of foreign substances, bacteria, or viruses that may occur from partial lifting on the scleral surface can be effectively prevented while maintaining a smooth outer curvature.

In one embodiment of the present invention, a specific cross section of the optically visible iris-pupil complex expansion part 105, which includes the anterior curved surface 120, the posterior inclined surface 110, and the inner annular edge 101 and the outer annular edge 102 where the anterior curved surface 120 and the posterior inclined surface 110 meet, can be designed in various dimensions and shapes according to a particular design specification. However, for example, in one embodiment of the present invention, the maximum-thickness portion t5 of the optically visible iris-pupil complex expansion part 105 may have a thickness of 50 μm to 200 μm (for example, about 120 μm), the edge of the inner annular edge 101 may have a thickness of 20 μm to 100 μm (for example, about 40 μm), and the edge of the outer annular edge 102 may have a thickness of 60 μm to 200 μm (for example, about 80 μm).

The thickness t₁ formed at the edge of the inner annular edge 101 may refer to the distance between the inner inflection point 101′ of the anterior curved surface 120 and the posterior inclined surface 110, when the anterior curved surface 120, which defines a thickness profile on the posterior inclined surface 110, meets the posterior inclined surface 110 along a steeper downward curve passing through the inner inflection point 101′ to form the edge of the inner annular edge 101. Similarly, the thickness t₂ formed at the edge of the outer annular edge 102 may refer to the distance between the outer inflection point 102′ of the anterior curved surface 120 and the posterior inclined surface 110, when the anterior curved surface 120, which defines a thickness profile on the posterior inclined surface 110, meets the posterior inclined surface 110 along a steeper downward curve passing through the outer inflection point 102′ to form the edge of the outer annular edge 102.

In one embodiment of the present invention, the optically visible iris-pupil complex expansion part 105 may include a profile of the anterior curved surface 120 that approaches the posterior inclined surface 110 from the maximum-thickness portion t5 toward both sides. The profile of the anterior curved surface 120 may contact the posterior inclined surface 110 along a steeper downward trajectory through the inner inflection point 101′ and the outer inflection point 102′, thereby forming the inner annular edge 101 and the outer annular edge 102. In one embodiment of the present invention, the distance from the outer annular edge 102 to the outer inflection point 102′ along the inclined surface direction may be longer than the distance from the inner annular edge 101 to the inner inflection point 101′. For example, in one embodiment of the present invention, the outer inflection point 102′ may be formed with a relatively greater thickness depth moving downward to form a relatively sharply rounded outer annular edge 102 from the anterior curved surface 120 having a relatively high average thickness. Conversely, the inner inflection point 101′ may be formed with a relatively smaller thickness depth moving downward to form a relatively gently rounded inner annular edge 101 from the anterior curved surface 120 having a relatively low average thickness. Accordingly, the distance from the outer inflection point 102′ to the outer annular edge 102 may be formed longer than the distance from the inner inflection point 101′ to the inner annular edge 101, reflecting the difference in curvature and thickness gradient between the inner and outer regions of the optically visible iris-pupil complex expansion part 105.

For example, in one embodiment of the present invention, the outer inflection point 102′ and the inner inflection point 101′ may refer to points at which the profile of the anterior curved surface 120 bends relatively sharply downward to form the outer annular edge 102 and the inner annular edge 101, respectively. Specifically, the two points with the greatest change in curvature along the profile of the anterior curved surface 120 may correspond to two downward-bending points located adjacent to the outer annular edge 102 and adjacent to the inner annular edge 101, respectively. In one embodiment of the present invention, the thickness t₂ of the edge of the outer annular edge 102 may be greater than the thickness t₁ of the edge of the inner annular edge 101. That is, the thickness t₂ corresponding to the distance moving downward from the outer inflection point 102′ to form a sharply rounded outer annular edge 102 from the outer area OA having a relatively high average thickness may be formed relatively larger.

For reference, throughout the present specification, the thicknesses of the outer area OA and the inner area IA, their average thicknesses, the thickness t₂ of the edge of the outer annular edge 102, and the thickness t₁ of the edge of the inner annular edge 101 correspond to thickness dimensions measured in the vertical direction from the posterior inclined surface 110. When expressing the relative relationship between these thickness dimensions, the terms “high” or “low” may be used in the same sense as for height dimensions. However, such expressions are for convenience of understanding, and in the context of thickness dimensions, they correspond to “thicker” or “thinner,” respectively.

In one embodiment of the present invention, the posterior inclined surface 110 may be formed to be inclined at a constant tilting angle θ from a vertical plane G perpendicular to a frontal direction Z3, along the frontal direction Z3 extending rearward from the anterior side of the eye including the optically visible iris-pupil complex. In addition, the posterior inclined surface 110 may be inclined at a tilting angle θ of approximately 20 to 40 degrees with respect to the vertical plane G. In one embodiment of the present invention, the posterior inclined surface 110 may be closely fitted to the sclera. For example, the posterior inclined surface 110 may be configured to adhere to a region of the sclera having a relatively small curvature that is closer to an inclined surface than to a curved surface, extending from the irregular edge DE to the curved portion AC of the sclera. In one embodiment of the present invention, the section of the sclera extending from the region beyond the irregular edge DE to the curved portion AC of the sclera may be regarded as an approximately inclined surface rather than a curved surface. Therefore, the posterior inclined surface 110 may be formed at a constant tilting angle θ on the vertical plane G so as to adhere to the portion of the sclera having a small curvature close to an inclined surface.

In one embodiment of the present invention, the posterior inclined surface 110 may be in close contact with the surface of the sclera that is substantially formed as or approximates an inclined surface. Accordingly, depending on the flexibility of the posterior inclined surface 110 or the ocular implant 100 including the same, the posterior inclined surface 110 may conform to and adhere closely to the scleral surface. In one embodiment of the present invention, the posterior inclined surface 110, being inclined at a constant tilting angle θ from the vertical plane G, may provide a support surface for the entire ocular implant 100. As described below, a packaging container for the ocular implant 100 according to one embodiment of the present invention may include an inclined support surface configured to support the posterior inclined surface 110. Considering the ease of manufacturing of both the ocular implant 100 and the packaging container of the ocular implant 100, the posterior support surface of the ocular implant 100 and the support surface of the packaging container may be formed to follow the same constant inclined profile, thereby providing stable and firm support there between.

In one embodiment of the present invention, for example, the inner annular edge 101 and the outer annular edge 102 each appear as an ellipse and a circle when viewed along a frontal direction Z3 from the anterior portion of the eyeball EB, where a crystalline lens that performs a lens function and a pupil forming a light entrance aperture are located, toward a posterior portion of the eyeball EB. The posterior inclined surface 110 may be formed at a constant tilting angle θ from a vertical plane G perpendicular to the frontal direction Z3, such that the inner annular edge 101 and the outer annular edge 102 define an anterior position and a posterior position, respectively.

According to one embodiment of the present invention, the ocular implant 100 may take a configuration in which, when viewed from the frontal direction Z3, the inner annular edge 101 located at the anterior position and the outer annular edge 102 located at the posterior position partially overlap according to the inclination of the posterior inclined surface 110 that provides a thickness reference, such that the elliptical shape of the inner annular edge 101 is encompassed within the circular shape of the outer annular edge 102. At this time, it is preferable that the optically visible iris-pupil complex expansion part 105 between the inner annular edge 101 and the outer annular edge 102 has a wider width at one side than at the opposite side along the minor-axis direction Z2 of the inner annular edge 101. To this end, the elliptical inner annular edge 101 may be positioned eccentrically within the circular outer annular edge 102, biased toward one side within the circle. As described below, one side of the optically visible iris-pupil complex expansion part 105 having a relatively wider width along the minor-axis direction Z2 of the elliptical inner annular edge 101 may correspond to a central position S of the surgical procedure. At the one-side position of the optically visible iris-pupil complex expansion part 105, an incision C may be provided, and first and second ends E₁ and E₂ separated from each other by the incision C may be formed. A surgical hole 10, a catch-engaging or hook-engaging connection for joining the first and second ends E₁ and E₂ toward each other may also be provided at the same position.

In one embodiment of the present invention, the inner annular edge 101 and the outer annular edge 102 may surround the opening OP along different contours. For example, the inner annular edge 101 may be formed into an elliptical shape along the contour of the optically visible iris-pupil complex so as to surround the outer rim of the optically visible iris-pupil complex at an adjacent position, thereby providing an appearance integrated with the optically visible iris-pupil complex without obscuring the optically visible iris-pupil complex forming the light entrance aperture. The outer annular edge 102 may be formed into a circular shape to provide a smoothly circular appearance. For example, the inner annular edge 101 may be formed as an ellipse having different major and minor axis lengths, L₁ and L₂, respectively. In this case, the major axis length L₁ may extend in a direction along which an implant recipient's pair of eyes face each other, while the minor axis length L₂ may extend in a direction perpendicular to the major axis length L₁.

As such, in one embodiment of the present invention, the inner annular edge 101 that surrounds the outer rim of the optically visible iris-pupil complex of the implant recipient may be formed into an elliptical shape corresponding to the natural shape of the implant recipient's optically visible iris-pupil complex. The inner annular edge 101 may also be variably deformed into an optimized elliptical configuration according to the individual physiological conditions of each implant recipient, such as the differential shape and size of the optically visible iris-pupil complex. For example, in an adult, the major axis length L₁ of the optically visible iris-pupil complex may range from 9 mm to 15 mm (e.g., 12 mm), and the minor axis length L₂ may range from 8 mm to 14 mm (e.g., 11 mm). Depending on the aspect ratio between the major axis length L₁ and the minor axis length L₂, the optically visible iris-pupil complex shape may be formed as either a slit-type ellipse, in which the major axis length L₁ is relatively longer than an average shape, or a rounded ellipse, in which the minor axis length L₂ is relatively longer than an average shape.

Hereinafter, the ocular implant 100 according to another aspect of the present invention will be described. The ocular implant 100 includes the adaptive stretchable part 151 that adaptively provides longitudinal stretching along the perimeter of the inner annular edge 101, and roughened surfaces 152 and 153 that generate rotational resistance in a rotational direction along the perimeter of the optically visible iris-pupil complex expansion part 105 formed between the inner annular edge 101 and the outer annular edge 102.

FIG. 6 is a perspective view describing the adaptive stretchable part 151 applied to the ocular implant 100 according to one embodiment of the present invention.

FIG. 7 is a perspective view describing the adaptive stretchable part 151 applied to the ocular implant 100 according to another embodiment of the present invention, and is a drawing for describing a modified example of FIG. 6.

FIGS. 8A and 8B are cross-sectional views of the ocular implant 100 taken along the circumferential direction of the optically visible iris-pupil complex expansion part 105, and are drawings illustrating different roughened surfaces 152 and 153 having isotropic rotational resistance and anisotropic rotational resistance, respectively.

The ocular implant 100 according to the above aspect may be permanently implanted on the eyeball EB of an implant recipient to provide an enlarged appearance to the implant recipient's optically visible iris-pupil complex.

The ocular implant 100 may include:
the central opening OP configured to receive light incident toward the pupil that provides the optical aperture of the eyeball EB;
the inner annular edge 101 defining the opening OP while surrounding the opening OP;
the outer annular edge 102 disposed opposite the inner annular edge 101 that defines the opening OP and surrounds the opening OP together with the inner annular edge 101; and
the adaptive stretchable part 151 that provides adaptive longitudinal stretching along the perimeter of the inner annular edge 101.

For example, the adaptive stretchable part 151 may provide longitudinal extensibility along the circumference of the inner annular edge 101 so as to adaptively deform according to the shape and size of the optically visible iris-pupil complex of each implant recipient.

For example, the inner annular edge 101 may be formed into an elliptical shape having different major and minor axis lengths, L₁ and L₂, respectively.

The adaptive stretchable part 151 may provide longitudinal extensibility along the major-axis direction Z1 and/or the minor-axis direction Z2 so as to adaptively provide the inner annular edge 101, which may have an elliptical shape with different major and minor axis lengths L₁ and L₂, in a form suitable for the optically visible iris-pupil complex of the implant recipient. Specifically, the inner annular edge 101 may be adaptively deformed into a slit-type elliptical shape, in which the major axis length L₁ is relatively elongated and/or the minor axis length L₂ is shortened, or into a rounded elliptical shape, in which the major axis length L₁ is relatively shortened and/or the minor axis length L₂ is elongated.

For example, the adaptive stretchable part 151 may include at least one slit 151s formed along the perimeter of the inner annular edge 101.

In another example, the adaptive stretchable part 151 may include a plurality of slits 151s formed at intervals along the perimeter of the inner annular edge 101.

For example, the plurality of slits 151s may be formed along the perimeter of the inner annular edge 101, spaced apart from each other at uniform intervals.

For example, the adaptive stretchable part 151 may include one or more groups of slits 151s formed at opposite positions along the major-axis direction Z1 and/or at opposite positions along the minor-axis direction Z2.

For example, the adaptive stretchable part 151 may be configured as follows:
through first and second groups of slits 151s formed at opposite positions along the minor-axis direction Z2, segmented portions divided from each other overlap while elongating the minor axis length L₂ and shortening the major-axis direction Z1; and
through third and fourth groups of slits 151s formed at opposite positions along the major-axis direction Z1, segmented portions divided from each other overlap while elongating the major axis length L₁ and shortening the minor axis length L₂.

For example, the slits 151s may be formed at angularly spaced positions with a constant interval, using the center O of the opening OP where the major axis length L₁ and the minor axis length L₂ of the inner annular edge 101 intersect as a rotation center.

For example, the slits 151s may include:
first and second groups of slits 151s formed at 0-degree and 180-degree angular positions A1 and A2, respectively, corresponding to opposite positions along the minor-axis direction Z2; and
third and fourth groups of slits 151s formed at 90-degree and 270-degree angular positions A3 and A4, respectively, corresponding to opposite positions along the major-axis direction Z1.

For example, the slits 151s may extend into the interior of the optically visible iris-pupil complex expansion part 105 along a depth direction from the inner annular edge 101 to the outer annular edge 102.

For example, the slits 151s may extend into the interior of the optically visible iris-pupil complex expansion part 105 in the depth direction along a radial direction from the center O of the opening OP, where the major axis length L₁ and the minor axis length L₂ of the inner annular edge 101 intersect.

For example, the adaptive stretchable part 151 may extend the length of the inner annular edge 101 as segmented portions divided by the slits 151s along the perimeter of the inner annular edge 101 overlap or slide relative to each other.

For example, the optically visible iris-pupil complex expansion part 105 may include:
the posterior inclined surface 110 disposed toward the eyeball EB so as to closely adhere to the surface of the eyeball EB; and
the anterior curved surface 120 disposed toward the external space OS opposite to the eyeball EB and forming a thickness profile with reference to the posterior inclined surface 110. The anterior curved surface 120 contacts the posterior inclined surface 110 at a position adjacent to the opening OP to form the inner annular edge 101, and also contacts the posterior inclined surface 110 at a position opposite the opening OP to form the outer annular edge 102. With reference to the posterior inclined surface 110, the anterior curved surface 120 defines a thickness profile such that, based on a maximum-thickness portion t₅ having the maximum thickness between the inner annular edge 101 and the outer annular edge 102, the relatively thin inner area IA and the relatively thick outer area OA are formed on opposite sides of the maximum-thickness portion t₅.

The segments divided through the slits 151s forming the adaptive stretchable part 151 may overlap or slide relative to each other to expand the length of the inner annular edge 101. Through this overlapping motion, the slits 151s extending inward from the inner annular edge 101 may form an additional thickness region in the inner area IA, thereby locally increasing the thickness of the inner area IA.

For example, the adaptive stretchable part 151 may be formed on the inner annular edge 101, which surrounds the outer ring of the optically visible iris-pupil complex, at a position relatively closer to the outer annular edge 102, so as to adaptively deform according to the shape and size of the optically visible iris-pupil complex of each implant recipient.

To maintain a smooth and aesthetically circular appearance against the deformation of the inner annular edge 101, the adaptive stretchable part 151 may not be formed on the outer annular edge 102.

For example, the inner annular edge 101, on which the segmented pieces overlap each other through the slits 151s forming the adaptive stretchable part 151, may be formed with a relatively gently rounded edge.

In contrast, the outer annular edge 102 may be formed with a relatively sharply rounded edge.

In the ocular implant 100 according to one embodiment of the present invention, the shape and size of the optically visible iris-pupil complex, which serves as the enlargement target, may be differentially determined according to the physical condition of the implant recipient. Furthermore, the inner annular edge 101 may absorb variations in the shape and size of the optically visible iris-pupil complex according to the individual physical characteristics of the implant recipient, so as to form an enlarged optically visible iris-pupil complex integrated with the natural optically visible iris-pupil complex without obstructing the optically visible iris-pupil complex that defines the light entrance aperture. To this end, the ocular implant 100 may include the adaptive stretchable part 151 capable of providing adaptive longitudinal stretching along the circumferential direction of the inner annular edge 101. For example, in one embodiment of the present invention, the adaptive stretchable part 151 may provide longitudinal stretching along the perimeter of the inner annular edge 101 to enable adaptive deformation corresponding to the shape and size of each implant recipient's optically visible iris-pupil complex.

In the present specification, the expression that the adaptive stretchable part 151 provides adaptive longitudinal stretching along the circumference of the inner annular edge 101 may include providing elongation along one of the major-axis direction Z1 and the minor-axis direction Z2 of the elliptical inner annular edge 101, or providing contraction along the other direction. It may also include elongating or contracting the entire circumference of the inner annular edge 101 without being limited to any specific direction.

In one embodiment of the present invention, the adaptive stretchable part 151 may include a plurality of slits 151s formed in the inner annular edge 101 or in a region of the optically visible iris-pupil complex expansion part 105 adjacent to the inner annular edge 101. For example, the adaptive stretchable part 151 may include at least one slit 151s formed along the perimeter of the inner annular edge 101. In one embodiment of the present invention, the adaptive stretchable part 151 may include a plurality of slits 151s spaced apart from each other along the circumferential direction of the inner annular edge 101 and extending inward from the inner annular edge 101 toward the optically visible iris-pupil complex expansion part 105.

For example, in the case of a practitioner performing implantation of the ocular implant 100, it is preferable to use the ocular implant 100 designed to be universally applicable yet capable of adaptively stretching to fit the individual shape and size of each implant recipient's optically visible iris-pupil complex, rather than manufacturing each implant in a fully customized manner for every individual. During implantation, such the ocular implant 100 can adaptively expand or contract to conform to the implant recipient's optically visible iris-pupil complex shape and size, thereby providing the ocular implant 100 optimized for the variously shaped and sized optically visible iris-pupil complex formed in different individuals. To this end, instead of introducing separately formed molds to manufacture individualized ocular implants 100, a plurality of ocular implants 100 may be formed using a single universal mold design, which reduces manufacturing costs and thus enables an economically efficient product design.

In one embodiment of the present invention, the adaptive stretchable part 151 may include a plurality of slits 151s spaced apart from each other along the perimeter of the inner annular edge 101. Each slit 151s may extend inward from the corresponding position on the inner annular edge 101 toward the optically visible iris-pupil complex expansion part 105. For example, in one embodiment of the present invention, the inner annular edge 101 may correspond to an innermost structure surrounding the outer ring of the optically visible iris-pupil complex in the ocular implant 100, either directly encircling the outer ring of the optically visible iris-pupil complex or forming a region where elastic deformation is induced to achieve a design optimized for the shape and size of each implant recipient's optically visible iris-pupil complex. In one embodiment of the present invention, the inner annular edge 101 is preferably designed to allow a high degree of deformation freedom, enabling optimization according to the optically visible iris-pupil complex shape and size of each implant recipient. That is, the inner annular edge 101 may surround optically visible iris-pupil complex of various shapes and sizes at their closest positions without obscuring the optically visible iris-pupil complex corresponding to the light entrance aperture. To this end, deformation of the inner annular edge 101, such as shape distortion or overall elongation and contraction, may be induced through the adaptive stretchable part 151. Throughout the present specification, the term “longitudinal stretching of the inner annular edge 101” may comprehensively include both shape deformation, such as distortion of the inner annular edge 101, and overall elongation or contraction of the inner annular edge 101.

In one embodiment of the present invention, the adaptive stretchable part 151 may include a plurality of slits 151s formed to be spaced apart from each other along the perimeter of the inner annular edge 101. For example, the plurality of slits 151s may be formed at equal intervals along the perimeter of the inner annular edge 101. In one example, the slits 151s may be uniformly arranged at angular positions spaced apart by a constant angle around the center O of the opening OP serving as a rotational center.

In one embodiment of the present invention, the slits 151s forming the adaptive stretchable part 151 may extend into the interior of the optically visible iris-pupil complex expansion part 105 along a depth direction from the inner annular edge 101 to the outer annular edge 102. More specifically, each slit 151s forming the adaptive stretchable part 151 may be formed so as to extend inward from the inner annular edge 101 toward the interior of the optically visible iris-pupil complex expansion part 105. For example, each slit 151s may extend from a corresponding position of the inner annular edge 101 in a direction perpendicular to the inner annular edge 101, and may extend toward the interior of the optically visible iris-pupil complex expansion part 105. In various embodiments of the present invention, the slits 151s may extend perpendicularly from the inner annular edge 101 or may extend radially from the inner annular edge 101. For example, in various embodiments of the present invention, the slits 151s may extend in the radial depth direction toward the center O of the opening OP surrounded by the inner annular edge 101, thereby extending into the interior region of the optically visible iris-pupil complex expansion part 105 adjacent to the inner annular edge 101. For example, throughout the present specification, the term “center O of the opening OP surrounded by the inner annular edge 101” may refer to the center O of the opening OP where the major axis length L₁ and the minor axis length L₂ of the elliptical inner annular edge 101 (or of the elliptical opening OP) intersect. However, in various embodiments of the present invention, the slits 151s may extend into the interior of the optically visible iris-pupil complex expansion part 105 along various directions from the inner annular edge 101, and the depth direction in which the slits 151s extend may be variably set according to the shape formed by the inner annular edge 101, for example, the elliptical shape formed by the inner annular edge 101, and, as described below, according to the deformation of the inner annular edge 101 induced by the individual physical characteristics of the implant recipient.

In one embodiment of the present invention, the slits 151s forming the adaptive stretchable part 151 may be formed in a range of 30 to 360 slits along the perimeter of the inner annular edge 101. For example, when the slits 151s are arranged at uniform angular intervals, approximately 30 slits may be formed at an interval of about 12 degrees, or approximately 360 slits may be formed at an interval of about 1 degree. In addition, a width d of each slit 151s forming the adaptive stretchable part 151 may be formed to be approximately ¼ to ½ of the width of the optically visible iris-pupil complex expansion part 105 between the inner annular edge 101 and the outer annular edge 102 along the depth direction from the inner annular edge 101 to the outer annular edge 102.

In one embodiment of the present invention, the inner annular edge 101 may be formed into an elliptical shape having different major axis length L₁ and minor axis length L₂, and the adaptive stretchable part 151 may provide longitudinal stretching along the major-axis direction Z1 and/or the minor-axis direction Z2. Through this configuration, the adaptive stretchable part 151 may allow the inner annular edge 101 to adaptively assume either a slim-type elliptical shape having a relatively elongated major axis length L₁ and/or shortened minor axis length L₂, or a round-type elliptical shape having a relatively shortened major axis length L₁ and/or elongated minor axis length L₂, depending on the shape of the implant recipient's optically visible iris-pupil complex.

The inner annular edge 101 or the optically visible iris-pupil complex expansion part 105 adjacent thereto may be divided into a plurality of segments along the circumferential direction of the inner annular edge 101 through the respective slits 151s. A plurality of the segments, divided on both sides of each slit 151s along the circumferential direction of the inner annular edge 101, may overlap with one another, thereby enabling the length of the inner annular edge 101 to stretch. For example, in one embodiment of the present invention, the adaptive stretchable part 151 including an array of a plurality of slits 151s may be deformed such that the segments divided through the respective slits 151s approach and overlap each other through the slits 151s, thereby allowing the overall shape and length of the inner annular edge 101 to be adaptively extended.

In one embodiment of the present invention, the inner annular edge 101 to which the adaptive stretchable part 151 is applied, or the inner area IA having the inner annular edge 101 as a boundary, may be formed with a relatively low average thickness and may thus be capable of relatively free deformation. For example, the inner area IA having a relatively low average thickness may provide a clearance space in the thickness direction to accommodate the overlapping of the divided segments created through the slits 151s. In one embodiment of the present invention, the adaptive stretchable part 151 formed on the inner annular edge 101, together with the inner area IA having a relatively low average thickness and terminating at the inner annular edge 101, may cooperate to adaptively deform the inner annular edge 101 into a shape and size optimized according to the physical characteristics of the implant recipient. For example, the inner annular edge 101 formed with a relatively low average thickness may provide sufficient ductility to facilitate deformation, allowing the divided segments separated by the slits 151s to approach and overlap each other. Furthermore, the inner area IA formed with a relatively low average thickness may provide a spatial allowance that accommodates the additional thickness generated when the divided segments overlap each other through the slits 151s.

For example, in one embodiment of the present invention, the optically visible iris-pupil complex expansion part 105 may include: the posterior inclined surface 110 positioned toward the eyeball EB so as to closely adhere thereto; and the anterior curved surface 120 positioned toward the external space OS opposite to the eyeball EB, forming a thickness profile with reference to the posterior inclined surface 110. The anterior curved surface 120 may contact the posterior inclined surface 110 at a position adjacent to the opening OP to form the inner annular edge 101, and may contact the posterior inclined surface 110 at a position opposite to the opening OP to form the outer annular edge 102. The anterior curved surface 120 may further form a matching thickness profile with respect to the posterior inclined surface 110, such that, based on the maximum-thickness portion t5 forming the maximum thickness between the inner annular edge 101 and the outer annular edge 102, the relatively thin inner area IA and the relatively thick outer area OA are established. In addition, through the slits 151s forming the adaptive stretchable part 151, the divided segments may overlap each other, thereby stretching the length of the inner annular edge 101 and forming an extra thickness that increases the thickness of the inner area IA in which the slits 151s introduced from the inner annular edge 101 are formed.

In one embodiment of the present invention, the optically visible iris-pupil complex surrounded by the inner annular edge 101 may be formed into an elliptical shape having different major axis length L₁ and minor axis length L₂ when observed in the frontal direction Z3. The major axis length L₁ of the ellipse formed by the optically visible iris-pupil complex may be formed along a direction in which a pair of eyes face each other, and the minor axis length L₂ of the ellipse formed by the optically visible iris-pupil complex may be formed along a direction perpendicular to the major axis length L₁. For example, the major axis length L₁ of the ellipse formed by the optically visible iris-pupil complex may be 9 mm to 15 mm, and the minor axis length L₂ of the ellipse formed by the optically visible iris-pupil complex may be 8 mm to 14 mm. That is, the shape and size of the optically visible iris-pupil complex, which is to be enlarged by the ocular implant 100 according to one embodiment of the present invention, may have such variations. For example, even when the optically visible iris-pupil complex has different minor axis length L₂ and major axis length L₁, respectively, different elliptical shapes and different sizes may be formed due to the dispersion in the major axis length L₁ and the minor axis length L₂. For example, in one embodiment of the present invention, the shape and size of the inner annular edge 101 may need to be modified into a slim-type ellipse having a relatively longer major axis length L₁ and/or a relatively shorter minor axis length L₂ compared with an average shape and size, or into a near-round ellipse having a relatively longer minor axis length L₂ and/or a relatively shorter major axis length L₁, depending on the individual physical conditions of each implant recipient. Throughout the present specification, different elliptical shapes, such as the slim-type ellipse or the near-round ellipse, may be defined according to the relative aspect ratio between the major axis length L₁ and the minor axis length L₂, and may not necessarily mean the individual magnitude or absolute length of the major axis length L₁ and the minor axis length L₂. For example, when the major axis length L₁ is deformed to be relatively elongated or the minor axis length L₂ is deformed to be relatively elongated so as to be optimized to the differential shape and size of the recipient's optically visible iris-pupil complex, it may mean that deformation of the adaptive stretchable part 151 is induced so that the relative aspect ratio between the major axis length L₁ and the minor axis length L₂ defining the elliptical shape of the inner annular edge 101 surrounding the outer rim of the optically visible iris-pupil complex is changed.

In one embodiment of the present invention, the inner annular edge 101 surrounding the optically visible iris-pupil complex may be designed to match the shape and size of an average optically visible iris-pupil complex without requiring a separate adaptive stretchable part 151 or deformation in the inner area IA. A single-design ocular implant 100, capable of being universally applied, may thus be manufactured to fit the average optically visible iris-pupil complex shape and size without structural deformation. For example, in one embodiment of the present invention, the shape of the opening OP surrounded by the inner annular edge 101 may be elliptical, having a relatively longer major axis length L₁ and a relatively shorter minor axis length L₂. For instance, the major axis length L₁ and the minor axis length L₂ of the ellipse defining the central opening OP may be formed to 12 mm and 11 mm, respectively, to correspond to an average optically visible iris-pupil complex dimension.

In one embodiment of the present invention, a plurality of slits 151s may be formed along the circumference of the inner annular edge 101. The plurality of slits 151s may be uniformly formed along the entire perimeter of the inner annular edge 101. For example, the uniformly arranged slits 151s formed along the inner annular edge 101 may induce different deformation behaviors depending on the individual physical characteristics of each implant recipient. For instance, in one embodiment of the present invention, when the inner annular edge 101, originally having a non-deformed elliptical shape, is applied to a optically visible iris-pupil complex having a slim-type ellipse that requires an extension of the major axis length L₁, the segments of the inner annular edge 101 divided through the slits 151s formed at both sides along the major axis length L₁ may overlap, thereby extending the major axis length L₁ instead of shortening the minor axis length L₂. Conversely, when the inner annular edge 101 is applied to an optically visible iris-pupil complex having a round-type ellipse that requires an extension of the minor axis length L₂, the segments of the inner annular edge 101 divided through the slits 151s formed at both sides along the minor axis length L₂ may overlap, thereby extending the minor axis length L₂ instead of shortening the major axis length L₁.

In one embodiment of the present invention, deformation induced in the inner annular edge 101 can be flexibly absorbed by the inner annular edge 101, which is locally deformed into an optimized shape at each position where a slot is formed through uniformly distributed slots along the circumference of the inner annular edge 101. For example, according to the deformation induced in the inner annular edge 101, the deformation energy can be smoothly accommodated without generating concentrated stress. Depending on the magnitude and direction of deformation applied to the inner annular edge 101, the present embodiment can prevent damage to the ocular implant 100, including the inner annular edge 101, by avoiding distortion or loss of co-planarity that may occur due to an excess portion of the inner annular edge 101, and by preventing excessive stress or stress accumulation that may result from a deficient portion thereof. However, in various embodiments of the present invention, considering that maximum deformation may be required in the slits 151s formed at both sides of the major axis length L₁ and at both sides of the minor axis length L₂, depending on the shape of the optically visible iris-pupil complex such as a slim-type ellipse or a round-type ellipse, and further considering that the formation of an excessive number of uniformly distributed slits 151s along the inner annular edge 101 may reduce the overall rigidity of the ocular implant 100 and thereby decrease handling stability during distribution or surgery, the adaptive stretchable part 151 may include a plurality of slits 151s concentratedly formed at opposite positions along the major axis length L₁ of the inner annular edge 101, and a plurality of slits 151s concentratedly formed at opposite positions along the minor axis length L₂ of the inner annular edge 101. For example, when observed in the frontal direction Z3, the adaptive stretchable part 151 may include a plurality of slits 151s concentratedly arranged at angular positions 0° (A1), 90° (A3), 180° (A2), and 270° (A4) along the perimeter of the inner annular edge 101, so that major deformation regions along the inner annular edge 101 can be effectively accommodated by arranging multiple slits 151s at approximately 90-degree intervals. In one embodiment of the present invention, the adaptive stretchable part 151 may include a first group of slits 151s formed at the 0-degree angular position A1 and a second group of slits 151s formed at the 180-degree angular position A2, corresponding to opposite positions along the minor-axis direction Z2 of the inner annular edge 101, and these first and second groups of slits 151s, formed at the 0° and 180° positions, respectively, are arranged along the minor-axis direction Z2 to provide elongation of the minor axis length L₂ or shortening of the major axis length L₁. In addition, the adaptive stretchable part 151 may include a third group of slits 151s formed at the 90-degree angular position A3 and a fourth group of slits 151s formed at the 270-degree angular position A4, corresponding to opposite positions along the major-axis direction Z1 of the inner annular edge 101, and these third and fourth groups of slits 151s, formed at the 90° and 270° positions, respectively, are arranged along the major-axis direction Z1 to provide elongation of the major axis length L₁ or shortening of the minor axis length L₂.

In one embodiment of the present invention, a plurality of slits 151s forming the adaptive stretchable part 151 may be uniformly arranged at equal angular intervals along the circumference of the inner annular edge 101 with a constant intervening angle between adjacent slits. However, the first to fourth groups of slits 151s, which are arranged at approximately 90-degree intervals and contribute more significantly to the extension of the major axis length L₁ and the extension of the minor axis length L₂, or which are disposed at opposite positions along the major-axis direction Z1 and the minor-axis direction Z2, may be concentratedly formed around the 0-degree angular position A1, the 90-degree angular position A3, the 180-degree angular position A2, and the 270-degree angular position A4, respectively. For example, the first to fourth groups of slits 151s may be formed to a greater depth along the depth direction from the inner annular edge 101 to the outer annular edge 102 than the remaining slits 151s, or may be formed at a smaller intervening angle, thereby achieving higher local elasticity and deformation responsiveness.

For example, in one embodiment of the present invention, the adaptive stretchable part 151 may include: the first group of slits 151s concentratedly arranged around the 0-degree angular position A1; the second group of slits 151s concentratedly arranged around the 180-degree angular position A2; the third group of slits 151s concentratedly arranged around the 90-degree angular position A3; and the fourth group of slits 151s concentratedly arranged around the 270-degree angular position A4. Each of the first to fourth groups of slits 151s may be formed in different numbers, and may include at least one slit 151s. In various embodiments of the present invention, each of the first to fourth groups of slits 151s may include a single slit 151s, such that one slit 151s is formed respectively at the 0-degree position A1, 90-degree position A3, 180-degree position A2, and 270-degree position A4.

In various embodiments of the present invention, at angular positions corresponding to both sides along the major-axis direction Z1 and both sides along the minor-axis direction Z2, that is, at angular positions spaced at approximately 90-degree intervals where a relatively high degree of deformation is required, slits 151s extending to a relatively greater depth or slits 151s spaced at a relatively narrower angular interval may be formed. At the remaining angular positions, slits 151s inserted to a relatively shallower depth or slits 151s arranged at a relatively wider angular interval may be formed.

In one embodiment of the present invention, the adaptive stretchable part 151 may not be formed on the outer annular edge 102. For example, in one embodiment of the present invention, the adaptive stretchable part 151 formed on the inner annular edge 101 is designed in consideration of the individual differences in the shape and size of the optically visible iris-pupil complex. The adaptive stretchable part 151 is provided on the inner annular edge 101 surrounding the optically visible iris-pupil complex so that a single-design ocular implant 100 or a single-design mold can be universally applied, thereby significantly reducing the manufacturing cost while allowing permanent implantation in a form optimized for the individual physical characteristics of each implant recipient by inducing deformation suitable for each person. In contrast, the outer annular edge 102 may not include any structure for inducing or facilitating deformation. For example, if a structure advantageous to deformation, such as a slit 151s, were formed on the outer annular edge 102, unintended deformation of the outer annular edge 102 following the deformation induced in the inner annular edge 101 could distort the originally designed circular appearance, damaging the aesthetic appearance imparted to the outer annular edge 102. Therefore, by not forming a deformation-facilitating structure such as a slit 151s on the outer annular edge 102 that would otherwise follow the deformation induced in the inner annular edge 101, the originally designed beautiful circular appearance can be maintained. In one embodiment of the present invention, the outer annular edge 102 may be formed in a circular shape to enhance aesthetic satisfaction. Although deformation may be induced in the inner annular edge 101, for example, deformation to match a slim-type oval optically visible iris-pupil complex or a round-type oval optically visible iris-pupil complex, the outer annular edge 102 can maintain its circular appearance because no deformation-facilitating structure (such as the adaptive stretchable part 151) or slit 151s is formed on the outer annular edge 102.

In one embodiment of the present invention, the outer annular edge 102 may be formed with higher rigidity than the inner annular edge 101, and may have a relatively greater average thickness than the inner annular edge 101 so as to provide a difference in rigidity through a differential shape design, while being made of the same material. Accordingly, in one embodiment of the present invention, the outer annular edge 102 may exhibit higher shape stability and shape retention capability than the inner annular edge 101, and may maintain a beautiful circular shape even when deformation is induced in the inner annular edge 101.

In the ocular implant 100 according to one embodiment of the present invention, the optically visible iris-pupil complex expansion part 105 forming the body of the ocular implant 100 may include the posterior inclined surface 110 directed toward the eyeball EB and the anterior curved surface 120 directed toward the external space OS opposite to the eyeball EB. In one embodiment of the present invention, the term “external space OS” opposite to the eyeball EB does not refer to the external environment of the human body, but rather to the outer environment of the eyeball EB itself. For example, a body tissue such as an eyelid that covers the front of the eyeball EB may also correspond to the external space OS of the eyeball EB.

The ocular implant 100 may be implanted onto the sclera such that the posterior inclined surface 110 is in close contact with the scleral surface, and may be formed with roughened surfaces 152 and 153 having an appropriate surface roughness to generate suitable friction with the sclera. In other words, the posterior inclined surface 110 and the anterior curved surface 120 may be formed as different levels of surface roughness. Specifically, the posterior inclined surface 110 may be formed as roughened surfaces 152 and 153 having an appropriate roughness capable of inducing frictional contact with the sclera, while the anterior curved surface 120 may be formed as a smooth surface to provide an aesthetically pleasing external appearance.

In one embodiment of the present invention, the ocular implant 100 may be formed in a rotationally asymmetric shape, rather than a rotationally symmetric shape. For example, in the ocular implant 100, the inner annular edge 101 surrounding the optically visible iris-pupil complex may be formed in an elliptical shape having different major axis length L₁ and minor axis length L₂. The ocular implant 100 formed in such a rotationally asymmetric shape may be rotated arbitrarily on the spherical eyeball EB. For example, since the position of the ocular implant 100 is difficult to adjust after permanent implantation, the posterior inclined surface 110 disposed to face the eyeball EB may be formed with roughened surfaces 152 and 153 having an appropriate surface roughness to induce friction, thereby maintaining the orientation of the major axis length L₁ and the minor axis length L₂ and preventing unintended rotation on the eyeball EB. In one embodiment of the present invention, the posterior inclined surface 110 may be formed in any shape capable of inducing friction, and may include any concave-convex pattern capable of forming surface roughness. In another embodiment of the present invention, the posterior inclined surface 110 may include roughened surfaces 152 and 153 in which a continuously repeated concave-convex pattern is formed, such as a wave-like shape. In various embodiments of the present invention, the roughened surface 152 may be formed to have isotropic rotational resistance, inducing substantially identical friction in both a rotational direction and an opposite rotational direction. Alternatively, the roughened surface 153 may be formed to have anisotropic rotational resistance, inducing different frictional forces in a rotational direction and an opposite rotational direction.

For example, during the implantation of the ocular implant 100, the roughened surface 153 may provide anisotropic rotational resistance such that, when the ocular implant 100 is inserted through the implantation space between the conjunctiva and the sclera, which is secured from an incision EBC formed on the conjunctiva covering the sclera, a relatively low rotational resistance acts along the implantation direction through the roughened surface 153 providing the anisotropic rotational resistance, whereas, once implanted, when the ocular implant 100 attempts to rotate arbitrarily in a direction opposite to the implantation direction, a relatively high rotational resistance acts through the same roughened surface 153. That is, the anisotropic rotational resistance is provided such that a low rotational resistance is generated along the implantation direction, and a high rotational resistance is generated along the rotational direction opposite to the implantation direction. To this end, the roughened surface 153 may be formed in a waveform having directional anisotropy along the rotational direction. For example, along the rotational direction corresponding to the implantation direction, the roughened surface 153 may be formed with a relatively receding waveform having a gentle inclination to provide a low rotational resistance, and along the opposite rotational direction, the roughened surface 153 may be formed in a steeply inclined waveform to provide a high rotational resistance. In one embodiment of the present invention, the roughened surfaces 152 and 153 formed on the posterior inclined surface 110 may be formed along the circumference of the optically visible iris-pupil complex expansion part 105. For example, the ocular implant 100 may be implanted in one rotational direction so as to surround the outer ring of the optically visible iris-pupil complex, and by generating a relatively low rotational resistance along the implantation direction and a relatively high rotational resistance along the opposite rotational direction, the ocular implant 100 may be provided with a positional fixing force for maintaining its implantation position.

In this way, the inner annular edge 101 is preferably formed to reflect the natural shape of the optically visible iris-pupil complex at a position as close as possible to the optically visible iris-pupil complex, in consideration of the following: when the ocular implant is configured to surround the optically visible iris-pupil complex at a position that does not overlap with the optically visible iris-pupil complex so as not to interfere with the incidence of light directed toward the optically visible iris-pupil complex, namely, at a position surrounding the outer ring of the optically visible iris-pupil complex, if the inner annular edge 101 is excessively spaced apart from the optically visible iris-pupil complex, which forms a dark region of relatively low brightness, the implant may fail to provide an appearance visually integrated with the optically visible iris-pupil complex, thereby reducing the satisfaction with the appearance of the enlarged optically visible iris-pupil complex. To this end, in one embodiment of the present invention, the inner annular edge 101 may be formed in an elliptical shape reflecting the actual shape of the optically visible iris-pupil complex, and may surround the outer ring of the optically visible iris-pupil complex in an elliptical shape optimized for the individual optically visible iris-pupil complex of the implant recipient, by means of the adaptive stretchable part 151 formed on the inner annular edge 101. At this time, the outer annular edge 102 may be formed in a circular shape so that the outer contour of the enlarged optically visible iris-pupil complex appears circular. Unlike the inner annular edge 101, the outer annular edge 102 does not interfere with the light entering the optically visible iris-pupil complex, but rather defines the perceived outer contour of the enlarged optically visible iris-pupil complex and is thus preferably formed circular to provide a more aesthetically pleasing external appearance.

In one embodiment of the present invention, a plurality of slits 151s forming the adaptive stretchable part 151 may be formed along the perimeter of the inner annular edge 101, and the pieces divided through the plurality of slits 151s may overlap each other to provide elongation along the circumferential direction of the inner annular edge 101. At this time, the inner annular edge 101, where the slits 151s are formed, may be formed with a relatively gently rounded edge shape so that the pieces divided through the slits 151s can easily overlap one another. For example, the inner annular edge 101 may be formed with a relatively gently rounded edge shape, compared to the outer annular edge 102, which may be formed with a relatively sharply rounded edge shape.

For example, the inner annular edge 101 or a piece of the inner annular edge 101 divided through the slits 151s having the relatively gently rounded edge shape may provide a slightly lifted gap from the bottom through the gently rounded contour. Accordingly, the piece can easily slide under and overlap an adjacent piece through the lifted gap, thereby enabling smooth longitudinal stretching of the inner annular edge 101.

Hereinafter, in the ocular implant 100 according to another aspect of the present invention, the surgical hole 10, a catch-engaging or hook-engaging connection for binding the first and second ends E₁ and E₂, which are separated from each other through the incision C, toward each other, will be described.

FIG. 9 is a plan view of the ocular implant 100 as viewed from the front, illustrating the surgical hole 10 applied to the ocular implant according to one embodiment of the present invention.

FIGS. 10A to 10C are perspective views illustrating different ocular implants 100 according to various embodiments of the present invention, and are drawings for describing the catch-engaging or hook-engaging connection between the first and second slits 154a, 154b, 155a and 155b applied to the ocular implant 100.

FIGS. 11A and 11B are different drawings for describing the catch-engaging or hook-engaging connection between the assembly slit 156a and the assembly hole 156b applied to the ocular implant 100 according to one embodiment of the present invention.

The ocular implant 100, according to the above-described aspects, is permanently implanted on the eyeball EB of the implant recipient so as to provide an enlarged external appearance of the implant recipient's optically visible iris-pupil complex.

The ocular implant 100 may include the following:
the central opening OP configured to receive incident light toward the optically visible iris-pupil complex that provides an optical aperture of the eyeball EB;
the inner annular edge 101 defining the opening OP while surrounding the opening OP;
the outer annular edge 102 disposed opposite the inner annular edge 101 that defines the opening OP and surrounds the opening OP together with the inner annular edge 101; and
the optically visible iris-pupil complex expansion part 105 formed between the inner annular edge 101 and the outer annular edge 102, including first and second ends E₁ and E₂ separated from each other through the incision C, and bound toward each other through surgical holes 10 respectively formed therein.

For example, in the optically visible iris-pupil complex expansion part 105 between the inner annular edge 101 and the outer annular edge 102 may be formed such that one position along the minor-axis direction Z2 of the inner annular edge 101 has a greater width than the opposite position. To this end, the oval shape of the inner annular edge 101 may be positioned in an offset manner within the circular shape of the outer annular edge 102, biased toward the opposite side.

The incision C, the first and second ends E₁ and E₂ separated from each other through the incision C, and the surgical holes 10 formed on the first and second ends E₁ and E₂ may be located at one side of the optically visible iris-pupil complex expansion part 105 that is formed with a relatively wide width along the minor-axis direction Z2.

For example, in the optically visible iris-pupil complex expansion part 105 between the inner annular edge 101 and the outer annular edge 102,
positions on both sides along the minor-axis direction Z2 of the inner annular edge 101 may be formed with a relatively smaller curvature than positions on both sides along the major-axis direction Z1 of the inner annular edge 101.

The surgical hole 10 may be formed at one of the positions along the minor-axis direction Z2 having the relatively smaller curvature.

For example, the incision C may be formed along the minor-axis direction Z2 of the inner annular edge 101, and the first and second ends E₁ and E₂,
which are separated from each other through the incision C, may be joined to each other along the major-axis direction Z1 of the inner annular edge 101.

Each of the first and second ends E₁ and E₂ may be formed with a plurality of surgical holes 10 arranged along the major-axis direction Z1 of the inner annular edge 101.

For example, the surgical holes 10 may include:
a first surgical hole 11, through which a suture passes to bind the first and second ends E₁ and E₂ together; and
a second surgical hole 12, through which an implantation insertion tool penetrates the ocular implant 100 so that the ocular implant 100 can be positioned to circumnavigate the outer ring of the patient's optically visible iris-pupil complex through the conjunctival incision C on the sclera.

For example, the first surgical hole 11 may be formed with a relatively small diameter, and
the second surgical hole 12 may be formed with a relatively large diameter.

In addition, the first surgical hole 11 may be formed relatively close to the incision C.

The second surgical hole 12 may be formed relatively farther from the incision C.

Meanwhile, in another aspect according to the above-described embodiment, the ocular implant 100 is permanently implanted on the eyeball EB of an implant recipient to provide an enlarged appearance to the implant recipient's optically visible iris-pupil complex.

The ocular implant 100 may include:
the central opening OP configured to receive incident light toward the optically visible iris-pupil complex, which provides an optical aperture of the eyeball EB;
the inner annular edge 101 defining the opening OP while surrounding the opening OP;
the outer annular edge 102 disposed opposite the inner annular edge 101 that defines the opening OP and surrounds the opening OP together with the inner annular edge 101; and
the optically visible iris-pupil complex expansion part 105 formed between the inner annular edge 101 and the outer annular edge 102, the optically visible iris-pupil complex expansion part 105 including first and second ends E₁ and E₂ that are separated from each other through the incision C and joined with each other through the catch-engaging or hook-engaging connection.

For example, in the optically visible iris-pupil complex expansion part 105 between the inner annular edge 101 and the outer annular edge 102 may be formed such that one position along the minor-axis direction Z2 of the inner annular edge 101 has a greater width than the opposite position. To this end, the oval shape of the inner annular edge 101 may be positioned in an offset manner within the circular shape of the outer annular edge 102, biased toward the opposite side.

The incision C, the first and second ends E₁ and E₂ separated from each other through the incision C, and the catch-engaging or hook-engaging connection structure between the first and second ends E₁ and E₂ may be formed at one side of the ocular implant that is formed with a relatively wide width along the minor-axis direction Z2.

For example, the catch-engaging or hook-engaging connection structure may include first and second slits 154a, 154b, 155a and 155b respectively formed on the first and second ends E₁ and E₂.

For instance, the first and second slits 154a, 154b, 155a and 155b may include:
first portions 1541 and 1551 extending along the major-axis direction Z1 of the inner annular edge 101; and
second portions 1542 and 1552 extending from the first portions 1541 and 1551 toward the inner annular edge 101 or the outer annular edge 102 along the minor-axis direction Z2, or along an oblique direction following both the minor-axis direction Z2 and the major-axis direction Z1 simultaneously toward the inner annular edge 101 or the outer annular edge 102, and opening to the outside of the ocular implant 100.

For example, at least a portion of the first and second slits 154a, 154b, 155a and 155b may be formed in complementary shapes so as to form a catch-engaging or hook-engaging connection with each other and may be inter-fitted with one another.

For example, the first portions 1541 and 1551 of the first and second slits 154a, 154b, 155a and 155b may extend parallel to each other along the major-axis direction Z1 of the inner annular edge 101.

The second portions 1542 and 1552 of the first and second slits 154a, 154b, 155a and 155b may be formed in complementary shapes, such that the second portions 1542 and 1552 of one of the first and second slits 154a, 154b, 155a and 155b and the second portions 1542 and 1552 of the other extend in opposite directions, one toward the outer annular edge 102 and the other toward the inner annular edge 101, and are open to the outside of the ocular implant 100.

For example, the incision C may be formed along the minor-axis direction Z2 of the inner annular edge 101.

The first and second ends E₁ and E₂ may be arranged adjacent to each other along the major-axis direction Z1 of the inner annular edge 101.

Among the first and second slits 154a, 154b, 155a and 155b, one may include an array of multiple first slits 154a and 155a or multiple second slits 154b and 155b formed on the first end E₁ or the second end E₂ along the major-axis direction Z1, while the other may be formed with a single second slit 154b and 155b or a single first slit 154a and 155a.

The catch-engaging or hook-engaging connection structure may include:
an assembly slit 156a formed in the first end E₁; and
an assembly hole 156b formed in the second end E₂, into which the assembly slit 156a is inserted.

For example, an assembly guide unit 158 may be formed on the first end E₁ and positioned in front of the assembly slit 156a along the assembly direction between the first and second ends E₁ and E₂.

For instance, the assembly guide unit 158 may include:
a front-end protrusion 158a formed with a relatively narrow width at the front end of the first end E₁ along the assembly direction of the first and second ends E₁ and E₂; a rear end 158c positioned behind the front-end protrusion 158a and in front of the assembly slit 156a, and formed with a relatively wider width than a neck W₁ of the assembly slit 156a; and
a variable-width portion 158b connecting the differential widths between the front-end protrusion 158a and the rear end 158c, the variable-width portion 158b converging toward the front-end protrusion 158a along a diagonal direction that simultaneously follows the major-axis direction Z1 corresponding to the assembly direction and the minor-axis direction Z2 intersecting the major-axis direction Z1.

For example, the rear end 158c of the assembly guide unit 158 may be connected to a full width W₀ of the first end E₁ interposed between the assembly guide unit 158 and the assembly slit 156a.

For example, a cut line CL including an array of multiple perforations may be formed between the assembly guide unit 158 and the full width W₀ of the first end E₁.

For instance, the assembly slit 156a may be introduced from both sides of the inner annular edge 101 and the outer annular edge 102 along the minor-axis direction Z2 of the inner annular edge 101, thereby forming the neck W₁ having a minimum width.

When the neck W₁ of the assembly slit 156a is fitted into the assembly hole 156b on the second end E₂ side, separation between the first and second ends E₁ and E₂ may be prevented by the relatively wide full width W₀ of the first end E₁ formed before and after the assembly slit 156a.

According to one embodiment of the present invention, the ocular implant 100 may be implanted by forming an incision EBC in the conjunctiva surrounding the sclera, and by inserting the implant between the sclera and the conjunctiva through the incision EBC. In this state, the implant may be advanced between the sclera and the conjunctiva along the region corresponding to the outer ring of the optically visible iris-pupil complex so as to surround the outer ring of the optically visible iris-pupil complex along the interface between the sclera and the conjunctiva. In one embodiment of the present invention, the ocular implant 100 may be inserted in a rotational direction so as to circumferentially travel around the outer ring of the optically visible iris-pupil complex. For this purpose, the ocular implant 100 itself may be provided with an incision C formed in the implant body, so that the implant can be inserted along the rotational path of the outer ring of the optically visible iris-pupil complex. Rather than being formed as a continuous structure extending along the rotational direction, the ocular implant 100 may be configured in a separated form, in which the first and second ends E₁ and E₂ are disconnected from each other through the incision C formed at a specific angular position (for example, a 0-degree angular position A1) with the center O of the opening OP as the rotational center. Accordingly, the ocular implant 100 according to one embodiment of the present invention may include an opening OP that is open to the outside through the incision C, rather than a closed opening OP, and may include first and second ends E₁ and E₂ that are separated from each other with the incision C interposed there between.

In one embodiment of the present invention, the incision C may be formed along the minor-axis direction Z2 of the inner annular edge 101 or of the opening OP surrounded by the inner annular edge 101. The first and second ends E₁ and E₂, separated from each other through the incision C, may be joined toward each other along the major-axis direction Z1. In one embodiment of the present invention, the first and second ends E₁ and E₂ separated through the incision C may be joined toward each other along the major-axis direction Z1 through the surgical hole 10 formed on the first and second ends E₁ and E₂, or through the catch-engaging or hook-engaging connection as described below. At this time, the first and second slits 154a, 154b, 155a and 155b or the assembly slit 156a forming the above surgical hole 10 or the catch-engaging or hook-engaging connection structure may be arranged in plurality along the major-axis direction Z1 in which the first and second ends E₁ and E₂ extend.

In the present specification, the optically visible iris-pupil complex expansion part 105, in which the incision C and the first and second ends E₁ and E₂ are formed, refers to a structure in which the anterior curved surface 120 forms a thickness profile on the posterior inclined surface 110, based on the posterior inclined surface 110 that is tilted at a fixed tilt angle θ from the vertical plane G perpendicular to the frontal direction Z3 (that is, the optically visible iris-pupil complex expansion part 105 is not a flat structure parallel to the vertical plane G perpendicular to the frontal direction Z3). At this time, the major-axis direction Z1 and minor-axis direction Z2 may be defined with respect to the frontal direction Z3. However, for ease of understanding in the present specification, the posterior inclined surface 110 is assumed to be tilted at a fixed tilt angle θ such that the inner annular edge 101 is positioned at a front side, and the outer annular edge 102 is positioned at a rear side. Meanwhile, the optically visible iris-pupil complex expansion part 105, which is formed between the inner annular edge 101 positioned forward and the outer annular edge 102 positioned backward, may have a thickness profile that varies toward the anterior curved surface 120 formed on the posterior inclined surface 110, but generally has a shape inclined from front to rear. Accordingly, on the optically visible iris-pupil complex expansion part 105, the incision C may be formed along the minor-axis direction Z2 in alignment with the major-axis direction Z1 and the minor-axis direction Z2 defined in the frontal direction Z3, or the first and second ends E₁ and E₂ separated through the incision C may not necessarily be joined along the major-axis direction Z1 through the surgical hole 10 or the catch-engaging or hook-engaging connection. However, since the posterior inclined surface 110 is tilted at a relatively small angle of about 20° to 40° from the vertical plane G perpendicular to the frontal direction Z3, when the optically visible iris-pupil complex expansion part 105, on which the incision C, surgical hole 10, catch-engaging or hook-engaging connection is formed, is projected onto the vertical plane G, the directionality of these features (incision C, surgical hole 10, catch-engaging or hook-engaging connection, etc.) formed on the optically visible iris-pupil complex expansion part 105 can be sufficiently described by the major-axis direction Z1 and the minor-axis direction Z2 defined in the frontal direction Z3. For example, in one embodiment of the present invention, the major-axis direction Z1 and minor-axis direction Z2 may correspond to the lengthwise direction and widthwise direction, respectively, to form the first and second ends E₁ and E₂, or with respect to the incision C, surgical hole 10, catch-engaging or hook-engaging connection formed on the first and second ends E₁ and E₂, and may represent the lengthwise and widthwise directions of the first and second ends E₁ and E₂.

In one embodiment of the present invention, in the optically visible iris-pupil complex expansion part 105 between the inner annular edge 101 and the outer annular edge 102 may be formed such that one position along the minor-axis direction Z2 of the inner annular edge 101 has a greater width than the opposite position. To this end, the oval shape of the inner annular edge 101 may be positioned in an offset manner within the circular shape of the outer annular edge 102, biased toward the opposite side.

The incision C, the first and second ends E₁ and E₂ separated from each other through the incision C, and the surgical hole 10 or catch-engaging or hook-engaging connection structure for joining the first and second ends E₁ and E₂ together may be formed at the one side position having a relatively wide width along the minor-axis direction Z2. For example, the side position formed with a relatively wide width along the minor-axis direction Z2 may correspond to the central surgical position S during the implantation procedure of the ocular implant 100.

In the present specification, assuming that the optically visible iris-pupil complex expansion part 105 between the inner annular edge 101 and the outer annular edge 102 has first and second ends E₁ and E₂ separated through the incision C, the incision C and the first and second ends E₁ and E₂ separated through the incision C may be formed across the optically visible iris-pupil complex expansion part 105 and extend through both the inner annular edge 101, which defines one end of the optically visible iris-pupil complex expansion part 105, and the outer annular edge 102, which defines the other end of the optically visible iris-pupil complex expansion part 105. In this sense, the incision C may be said to define the first and second ends E₁ and E₂, which separate the ocular implant 100 into two adjoining portions.

In one embodiment of the present invention, in the optically visible iris-pupil complex expansion part 105 formed between the inner annular edge 101 and the outer annular edge 102, the positions on both sides along the minor-axis direction Z2 of the inner annular edge 101 may be formed with a relatively smaller curvature than the positions on both sides along the major-axis direction Z1 of the inner annular edge 101. Among these two side positions, the one having a relatively wider width may be selectively designated as the central surgical position S, and at this central surgical position S, corresponding to the side position formed with a smaller curvature and a wider width, there may be formed structures such as the incision C, the first and second ends E₁ and E₂, the surgical holes 10, and the catch-engaging or hook-engaging connection.

In the implantation procedure of the ocular implant 100 according to one embodiment of the present invention, the first end E₁ of the ocular implant 100 may be inserted through an incision EBC formed on the conjunctiva and guided to circumferentially travel along the outer ring of the optically visible iris-pupil complex between the sclera and conjunctiva. The implant then returns to the incision site, where the first end E₁, withdrawn through the incision C, is joined to the second end E₂, which remains outside and was not inserted through the incision C. Through this joining, the ocular implant 100 encircling the outer ring of the optically visible iris-pupil complex can be permanently implanted. At this time, during the connection of the first and second ends E₁ and E₂, a suture may be passed through the surgical holes 10 formed in the first and second ends E₁ and E₂, and the two ends E₁ and E₂ may be fastened together by tying a knot of the suture that connects the surgical holes 10 of the first and second ends E₁ and E₂. In one embodiment of the present invention, at least one surgical hole 10 may be formed in each of the first and second ends E₁ and E₂. In various embodiments, a plurality of surgical holes 10 may be formed in each of the first and second ends E₁ and E₂. In various embodiments, a plurality of surgical holes 10 may be formed in the first and second ends E₁ and E₂ of the optically visible iris-pupil complex expansion part 105 forming the ocular implant 100 or the body of the ocular implant 100. The plurality of surgical holes 10 may be arranged at regular intervals along the major-axis direction Z1 of each of the first and second ends E₁ and E₂. For example, depending on the shape and size of the optically visible iris-pupil complex, which may vary among implant recipients according to their individual physiological characteristics, the connection between the first and second ends E₁ and E₂ may be achieved through the surgical hole 10 positioned at an optimized location among the plurality of surgical holes 10 formed in the first and second ends E₁ and E₂. For example, the surgeon performing the procedure of the ocular implant 100 may selectively utilize the surgical hole 10 formed in the first and second ends E₁ and E₂ located at a position most suitable for the individualized shape and size of each implant recipient's optically visible iris-pupil complex, thereby joining the first and second ends E₁ and E₂ through the surgical hole 10 positioned at an optimized location for circumferentially surrounding the outer ring of the optically visible iris-pupil complex along its perimeter.

For example, the surgeon may select a surgical hole 10 positioned at an optimal location for surrounding the outer ring of the implant recipient's optically visible iris-pupil complex and insert a suture through the selected surgical hole 10 to fasten the first and second ends E₁ and E₂ together. If the distance between the pair of surgical holes 10, through which the suture passes to fasten the first and second ends E₁ and E₂ together, is set excessively long, the optically visible iris-pupil complex expansion part 105 between the pair of surgical holes 10 may be distorted and deformed, adversely affecting the co-planarity of the entire optically visible iris-pupil complex expansion part 105. In addition, such distortion may cause side effects, such as the entire ocular implant 100 including the optically visible iris-pupil complex expansion part 105 failing to adhere closely to the sclera and forming an unintended gap. Conversely, if the distance between the pair of surgical holes 10, through which the suture passes to fasten the first and second ends E₁ and E₂ together, is set excessively short, excessive tensile stress may accumulate along the optically visible iris-pupil complex expansion part 105 or along the body of the ocular implant 100 formed by the optically visible iris-pupil complex expansion part 105 due to the suture tension acting between the surgical holes 10 used for fastening the first and second ends E₁ and E₂ together. The accumulated tensile stress may affect the durability and service life of the ocular implant 100 or of the optically visible iris-pupil complex expansion part 105 forming its body, or it may deform the optically visible iris-pupil complex expansion part 105 to reduce its circumferential length, thereby causing the optically visible iris-pupil complex expansion part 105 to cover or obstruct the recipient's optically visible iris-pupil complex, resulting in a critical surgical error. Alternatively, the optically visible iris-pupil complex expansion part 105 may undergo distortion and deformation, causing the optically visible iris-pupil complex expansion part 105 or the ocular implant 100 including the optically visible iris-pupil complex expansion part 105 to fail to closely adhere to the sclera of the recipient, thereby forming an undesired void and leading to adverse side effects.

In this manner, in one embodiment of the present invention, the ocular implant 100 may be formed with identical specifications using the same mold design, while still being adaptively optimized for the shape and size of the optically visible iris-pupil complex, which may vary depending on each implant recipient's physical characteristics. To achieve such optimization, among a plurality of surgical holes 10 formed in the first and second ends E₁ and E₂ of the ocular implant 100 that circumferentially travel around the outer ring of the optically visible iris-pupil complex, a binding position optimized for surrounding the implant recipient's optically visible iris-pupil complex may be selectively chosen. A suture may then be passed through the selected surgical hole 10 at the binding position taken from among the plurality of surgical holes 10, thereby joining the first and second ends E₁ and E₂ of the optically visible iris-pupil complex expansion part 105, which forms the ocular implant 100 or the body of the ocular implant 100, so as to circumferentially secure the ocular implant 100 around the implant recipient's optically visible iris-pupil complex. For example, in one embodiment of the present invention, the surgeon may cut and remove any excess portion of the optically visible iris-pupil complex expansion part 105 that remains between the paired surgical holes 10 selected as the binding positions at the first and second ends E₁ and E₂. The remaining excess portion of the optically visible iris-pupil complex expansion part 105 between the paired surgical holes 10 may be susceptible to deformation, as it is subjected to compressive stress generated by the tensile force of the suture passed through the surgical holes 10 selected as the binding positions, which tends to shorten the length between the tied holes. Accordingly, the excess portion of the optically visible iris-pupil complex expansion part 105 may be removed between the paired surgical holes 10 selected as the binding positions at the first and second ends E₁ and E₂. In this case, the surgeon may refer to the plurality of surgical holes 10 formed at regular intervals in the first and second ends E₁ and E₂, and determine the cutting position based on the positions of the surgical holes 10. In one embodiment of the present invention, the circumferential length of the optically visible iris-pupil complex expansion part 105 forming the ocular implant 100 or the body of the ocular implant 100 may preferably be set to a size optimized for surrounding the outer ring of the optically visible iris-pupil complex, so that the implanted optically visible iris-pupil complex expansion part 105 provides an appearance integrated with the individualized optically visible iris-pupil complex of each implant recipient while not obstructing the optically visible iris-pupil complex that serves as the light entrance opening. For example, by trimming the optically visible iris-pupil complex expansion part 105 formed to a standardized or single base size so as to form a circumferential length optimized to travel around the outer ring of the implant recipient's optically visible iris-pupil complex, a circumferential length optimized for surrounding the outer ring of the individualized optically visible iris-pupil complex of each implant recipient may be adaptively formed. In this case, the surgeon may refer to the positions of the surgical holes 10 formed at regular intervals in the first and second ends E₁ and E₂ of the ocular implant 100 or of the optically visible iris-pupil complex expansion part 105 forming the ocular implant 100 or the body of the ocular implant 100, and may cut at least a portion of one end of the first and second ends E₁ and E₂ so as to provide a circumferential length optimized for each implant recipient.

For example, the surgeon may determine the circumferential length of the ocular implant 100 or the optically visible iris-pupil complex expansion part 105 optimized to surround the optically visible iris-pupil complex of the implant recipient based on examination or measurement of the eyeball EB, and may set a cutting position from the end of at least one of the first and second ends E₁ and E₂ so as to form the determined circumferential length of the optically visible iris-pupil complex expansion part 105. In this case, the cutting position may be established based on the positions of a plurality of surgical holes 10 formed at regular intervals along the major-axis direction Z1 of the first and second ends E₁ and E₂. For example, in one embodiment of the present invention, the plurality of surgical holes 10 formed in each of the first and second ends E₁ and E₂ may be arranged at intervals of about 0.5 mm along the major-axis direction Z1.

In this manner, in one embodiment of the present invention, the plurality of surgical holes 10 formed along the major-axis direction Z1 or along the length of the first and second ends E₁ and E₂ can provide optimized binding positions for surrounding the outer ring of the optically visible iris-pupil complex that has an individualized shape and size for each implant recipient, and can also serve as references or guides for setting a cutting position to form a circumferential length optimized for surrounding the implant recipient's optically visible iris-pupil complex, for example, a circumferential length optimized for circling the outer ring of the optically visible iris-pupil complex.

In one embodiment of the present invention, the surgical hole 10 may serve as a passage through which an incision EBC is formed in the conjunctiva overlying the sclera, and the surgical hole 10 may further serve as an insertion path through which a space-securing instrument (not shown) is inserted via the incision EBC to secure an implantation space between the sclera and the conjunctiva for accommodating the ocular implant 100. The ocular implant 100 may then be drawn into the implantation space by means of an implant insertion device (not shown) so that the ocular implant 100 circumferentially travels along the outer ring of the optically visible iris-pupil complex while being guided within the secured implantation space. At this time, the implant insertion device (not shown) may be inserted through the surgical hole 10 formed in the first end E₁ of the ocular implant 100 and guided toward the implantation space defined along the outer ring of the optically visible iris-pupil complex of the implant recipient. During this process, as the implant insertion device (not shown) exerts a forced circumferential pulling motion, the ocular implant 100 may be inserted and guided from the first end E₁, through which the implant insertion device passes, into the implantation space along the outer ring of the optically visible iris-pupil complex, thereby circumferentially traveling around the optically visible iris-pupil complex's outer perimeter. Thus, the surgical hole 10 formed in the first or second end (E₁ or E₂) may provide a passage position for the implant insertion device (not shown), enabling traction and guidance of the entire ocular implant 100 during implantation. For example, the implant insertion device (not shown) fitted through the surgical hole 10 or through the first end E₁ in which the surgical hole 10 is formed may follow and be followed by the ocular implant 100 along the trajectory of the implant insertion device, so that the ocular implant 100 follows the movement of the insertion device and circumferentially travels around the outer ring of the optically visible iris-pupil complex of the implant recipient.

In one embodiment of the present invention, the surgical hole 10 may serve to define a binding position between the first and second ends E₁ and E₂ and may include the first surgical hole 11 for the passage of a suture and the second surgical hole 12 for physical engagement or penetration of an implant insertion device. In one embodiment of the present invention, the first and second surgical holes 11 and 12 constituting the surgical hole 10 may be formed with substantially the same shape and size. Depending on the surgeon's selection, some of the surgical holes 10 may function as first surgical holes 11 for the passage of a suture, while others may function as second surgical holes 12 for the passage of the implant insertion device (not shown). A plurality of surgical holes 10 formed with identical shapes along the major-axis direction Z1 or along the lengths of the first and second ends E₁ and E₂ may provide flexible positional selectivity, enabling the surgeon to freely choose the binding position during implantation. Accordingly, each surgical hole 10 may selectively function as either the first surgical hole 11 or the second surgical hole 12 according to the surgeon's choice during the procedure.

In various embodiments of the present invention, the surgical hole 10 may include the first surgical hole 11 configured to have a relatively small diameter to accommodate a fine suture strand, and the second surgical hole 12 configured to have a relatively large diameter to accommodate a thicker implant insertion device (not shown) for traction of the entire ocular implant 100. In one embodiment of the present invention, the first and second surgical holes 11 and 12 may be formed respectively at different positions along the first and second ends E₁ and E₂. The first surgical hole 11 for suture passage may be formed relatively close to the distal ends of the first and second ends E₁ and E₂ along the major-axis direction Z1. This configuration minimizes the possibility that excess portions of the ocular implant 100 or the optically visible iris-pupil complex expansion part 105 intervene between the paired first surgical holes 11 that define the binding position for joining the first and second ends E₁ and E₂. Meanwhile, the second surgical hole 12 may be formed at a position farther from the distal ends of the first and second ends E₁ and E₂ along the major-axis direction Z1. Accordingly, when the ocular implant 100 is drawn or guided by the implant insertion device (not shown) fitted into the second surgical hole 12, it is possible to avoid causing mechanical stress or damage to the first and second ends E₁ and E₂.

In one embodiment of the present invention, the ocular implant 100 may be formed in a symmetrical shape with respect to the center O of the opening OP. For example, in one embodiment of the present invention, the ocular implant 100, or the optically visible iris-pupil complex expansion part 105 forming the body of the ocular implant 100, may be symmetrically formed along both the major-axis direction Z1 and the minor-axis direction Z2, which intersect at the center O of the opening OP. In one embodiment of the present invention, the optically visible iris-pupil complex expansion part 105 forming the body of the ocular implant 100 may have an incision C formed along the minor-axis direction Z2, and a plurality of surgical holes 10 may be formed in the first and second ends E₁ and E₂ separated from each other through the incision C. The plurality of surgical holes 10 may be arranged along the major-axis direction Z1 and formed symmetrically with respect to the minor-axis direction Z2. For example, the plurality of surgical holes 10 formed in the first and second ends E₁ and E₂, separated by the incision C along the minor-axis direction Z2, may be symmetrically disposed with respect to the incision C. Accordingly, since the surgical holes 10 are symmetrically formed in the first and second ends E₁ and E₂ relative to the incision C, the surgeon can perform implantation without distinguishing between the first and second ends E₁ and E₂, thereby enhancing surgical convenience. In one embodiment of the present invention, the first and second surgical holes 11 and 12, having different sizes or diameters, may be symmetrically formed in the first and second ends E₁ and E₂. Because the second surgical holes 12, into which the implant insertion device is fitted, are symmetrically formed in both the first and second ends E₁ and E₂, the surgeon can initiate implantation of the ocular implant 100 through the implant insertion device fitted into the second surgical hole 12 of either one of the first and second ends E₁ and E₂.

In one embodiment of the present invention, the incision C may be formed along the minor-axis direction Z2 of the opening OP, and may be positioned at any one point along the minor-axis direction Z2 of the opening OP. A plurality of surgical holes 10 may be formed in the first and second ends E₁ and E₂, which are divided by the incision C formed at a selected position along the minor-axis direction Z2. The position of the first and second ends E₁ and E₂ where the surgical holes 10 are formed, or the position where the incision C dividing the first and second ends E₁ and E₂ is provided, may correspond to a region along the minor-axis direction Z2 of the opening OP, and may correspond to a portion of the ocular implant 100 or a portion of the optically visible iris-pupil complex expansion part 105 forming the body of the ocular implant 100, which extends parallel to the major-axis direction Z1.

In one embodiment of the present invention, the opening OP formed at the central position of the ocular implant 100, or the inner annular edge 101 defining the opening OP, may be formed in an elliptical shape having different major axis length L₁ and minor axis length L₂. The outer annular edge 102 formed opposite to the inner annular edge 101 may be formed in a circular shape to provide an aesthetically pleasing appearance. Accordingly, in one embodiment of the present invention, the optically visible iris-pupil complex expansion part 105 formed between the inner annular edge 101 and the outer annular edge 102 may have a configuration in which the elliptical inner annular edge 101 is positioned inside the circular outer annular edge 102. The optically visible iris-pupil complex expansion part 105 defined by the outer annular edge 102 and the inner annular edge 101 of different geometries may have different radial widths at different angular positions, with the central opening OP serving as the rotational center. For example, in one embodiment of the present invention, the optically visible iris-pupil complex expansion part 105 may have its maximum width at the 0-degree angular position A1, where the incision C is formed, and may have substantially equal widths at the remaining 90-degree (A3), 180-degree (A2), and 270-degree (A4) angular positions. In one embodiment of the present invention, the incision C, or the first and second ends E₁ and E₂ divided by the incision C, may correspond to the central position S of the implantation procedure of the ocular implant 100. For example, in the first and second ends E₁ and E₂, a suture may be inserted through a plurality of surgical holes (10, for example, first surgical holes 11) formed therein to join the first and second ends E₁ and E₂ toward each other. Alternatively, the ocular implant 100 may be implanted along the outer ring of the optically visible iris-pupil complex of the implant recipient by inserting an implant insertion device through the surgical holes (10, for example, second surgical holes 12) formed in the first and second ends E₁ and E₂.

In one embodiment of the present invention, in the optically visible iris-pupil complex expansion part 105 defined by the elliptical inner annular edge 101 and the circular outer annular edge 102, any one position along the minor-axis direction Z2 of the ellipse defined by the inner annular edge 101 and surrounded by the inner annular edge 101, that is, one of the 0-degree angular position A1 or the 180-degree angular position A2, which form opposite sides along the minor-axis direction Z2 with the center O of the opening OP as the rotational center, may be formed with a relatively wide width. For example, by forming the optically visible iris-pupil complex expansion part 105 (which constitutes the ocular implant 100 or the body of the ocular implant 100) so that it has the widest width at the 0-degree angular position A1, a procedure-center position S corresponding to this angular position (e.g., the 0-degree position A1) may be provided with a broadened region, thereby enhancing surgical accessibility and ease of implantation. Accordingly, in one embodiment of the present invention, the elliptical inner annular edge 101 is disposed inside the circular outer annular edge 102 such that the optically visible iris-pupil complex expansion part 105 has its maximum width at the position corresponding to the procedure-center position S (for example, at 0-degree position A1). Rather than being positioned at the exact geometric center of the circular outer annular edge 102, the elliptical inner annular edge 101 may be offset toward one side along the minor-axis direction Z2 so that a relatively wide width is secured at one side, while the opposite side remains relatively narrow. In other words, in one embodiment of the present invention, the elliptical inner annular edge 101 defining the opening OP may be positioned at a biased location along the minor-axis direction Z2 of the circular outer annular edge 102, such that the width at one side (e.g., 0-degree position A1) is increased relative to the opposite side (e.g., 180-degree position A2), thereby providing a structural offset advantageous for surgical manipulation and anchoring stability.

In one embodiment of the present invention, the position corresponding to the center position S of the procedure may correspond to one of the two positions on opposite sides along the minor-axis direction Z2, and in particular, to one of the two positions along the minor-axis direction Z2 having a relatively wide width. At the position corresponding to the center position S of the procedure, the incision C and the surgical hole 10 may be formed, and, as described below, a catch-engaging and a hook-engaging connection may also be provided at the same side position of the optically visible iris-pupil complex expansion part 105 along the minor-axis direction Z2.

Thus, in one embodiment of the present invention, one side position along the minor-axis direction Z2 may be formed with a relatively wide width, and may define a portion having a relatively small curvature while extending generally along the major-axis direction Z1. In contrast, positions on both sides along the major-axis direction Z1 may be formed with a relatively narrow width, and may define portions having a relatively large curvature while extending generally along the minor-axis direction Z2. Accordingly, by setting one side position along the minor-axis direction Z2 that defines a portion with a relatively small curvature as the procedure center position S, the ease of the surgical procedure can be improved. This improvement is achieved through the incision C formed in the portion of relatively wide width and small curvature, and through the surgical holes 10 formed in the first and second ends E₁ and E₂ that are divided by the incision C. At this side position corresponding to the procedure center position S along the minor-axis direction Z2, the incision C, the surgical holes 10, and, as will be described below, the catch-engaging or hook-engaging connection structure may all be formed together.

In one embodiment of the present invention, the first and second ends E₁ and E₂ may be fastened to each other by a suture thread inserted through the surgical holes 10 formed in the respective ends E₁ and E₂. However, in various embodiments of the present invention, the first and second ends E₁ and E₂ may alternatively form a catch-engaging or hook-engaging connection, in which one end of the first and second ends E₁ and E₂ is inserted into the other, thereby forming interlocking projections and recesses that prevent separation between the first and second ends E₁ and E₂ once joined.

More specifically, the first and second ends E₁ and E₂ may each be formed with first and second slits 154a, 154b, 155a and 155b, which serve as engagement channels for inserting the corresponding opposite ends. Through the first and second slits 154a, 154b, 155a and 155b, which are formed in complementary shapes to each other, the first and second ends E₁ and E₂ can be fitted together such that, once joined, disengagement between the first and second ends E₁ and E₂ through the complementarily formed first and second slits 154a, 154b, 155a and 155b may be prevented. The first and second ends E₁ and E₂ may thus be joined to each other through the first and second slits 154a, 154b, 155a and 155b.

In various embodiments of the present invention, the first and second slits 154a, 154b, 155a and 155b formed respectively in the first and second ends E₁ and E₂ may each include first portions 1541 and 1551 extending along the major-axis direction Z1, in which the first and second ends E₁ and E₂ face each other with the incision C interposed there between, or along the major-axis direction Z1 intersecting the minor-axis direction Z2 in which the incision C extends, and second portions 1542 and 1552 extending from the first portions 1541 and 1551 toward the outside of the ocular implant 100 or the optically visible iris-pupil complex expansion part 105 of the ocular implant 100 along the minor-axis direction Z2. In this case, the second portions 1542 and 1552 may be formed in complementary shapes to each other. For example, the second portions 1542 and 1552 of the first and second slits 154a, 154b, 155a and 155b formed respectively in the first and second ends E₁ and E₂ may open toward the outside of the optically visible iris-pupil complex expansion part 105 while extending in opposite directions along the minor-axis direction Z2 from the first portions 1541 and 1551, namely, extending respectively toward the inner annular edge 101 and the outer annular edge 102 that are disposed opposite each other from the first portions 1541 and 1551 and opening outward from the optically visible iris-pupil complex expansion part 105. When the ends are fitted together, the first and second slits 154a, 154b, 155a and 155b, of which the second portions 1542 and 1552 are formed in mutually complementary shapes, may function as catches or hooks that mechanically restrain separation between the both ends, thereby preventing disengagement between the corresponding regions of the optically visible iris-pupil complex expansion part 105. In various embodiments of the present invention, the first and second slits 154a, 154b, 155a and 155b may extend from the first portions 1541 and 1551, extending along the major-axis direction Z1, perpendicularly to the first portions 1541 and 1551 along the minor-axis direction Z2 toward the outside of the optically visible iris-pupil complex expansion part 105, thereby opening outwardly from the optically visible iris-pupil complex expansion part 105. Alternatively, the first portions 1541 and 1551 extending along the major-axis direction Z1 may be continued by the second portions 1542 and 1552 that extend obliquely outward, following a direction inclined relative to both the major-axis direction Z1 and the minor-axis direction Z2, and likewise open toward the outside of the optically visible iris-pupil complex expansion part 105.

In one embodiment of the present invention, at least one of the first and second slits 154a, 154b, 155a and 155b formed in the first and second ends E₁ and E₂ may be arranged in plurality along the major-axis direction Z1 or along the length direction of the first and second ends E₁ and E₂. In one embodiment of the present invention, the major-axis direction Z1, which is perpendicular to the minor-axis direction Z2 along which the incision C separating the first and second ends E₁ and E₂ extends, may correspond to the direction in which the plurality of first and second slits 154a, 154b, 155a and 155b or the plurality of surgical holes 10 are arranged. For example, at least one of the plurality of first and second slits 154a, 154b, 155a and 155b arranged in plurality may be used to adjust the binding position between the slits, thereby forming a circumferential length of the ocular implant 100 or of the optically visible iris-pupil complex expansion part 105 of the ocular implant 100 that is optimized for surrounding the optically visible iris-pupil complex of the implant recipient. The first and second ends E₁ and E₂ may thus be joined to each other through the first and second slits 154a, 154b, 155a and 155b at a selected position among the plurality of slits arranged along the length of the first and second ends E₁ and E₂.

In various embodiments of the present invention, the incision C may be formed along the minor-axis direction Z2 of the inner annular edge 101, and the first and second ends E₁ and E₂ may be disposed adjacent to each other along the major-axis direction Z1 of the inner annular edge 101. Among the first and second slits 154a, 154b, 155a and 155b, one of them may include an array of first slits 154a and 155a or an array of second slits 154b and 155b formed in plurality along the major-axis direction Z1 on the first end E₁ or the second end E₂, and the other may be formed with a single second slit 154b and 155b or a single first slit 154a and 155a.

In one embodiment of the present invention, in order to form a catch-engaging or hook-engaging connection structure that couples the first and second ends E₁ and E₂, an assembly slit 156a may be formed in one of the ends E₁ or E₂, and an assembly hole 156b, into which the assembly slit 156a is fitted, may be formed in the other. For example, in one embodiment of the present invention, the assembly slit 156a may be formed in the first end E₁, and the assembly hole 156b into which the assembly slit 156a is fitted may be formed in the second end E₂. In this embodiment, the assembly hole 156b formed on the second end E₂, into which the assembly slit 156a on the first end E₁ is inserted, may be configured in a closed structure that is isolated from the outside of the second end E₂ and not open toward the exterior surface thereof. Accordingly, in consideration of the ease of fitting assembly of the assembly slit 156a on the first end E₁, the assembly guide unit 158 may be provided at the tip of the first end E₁. The assembly guide unit 158 may include a rear end 158c having a relatively wide width and the front-end protrusion 158a having a relatively narrow width along the assembly direction or major-axis direction Z1, and may further include the variable-width portion 158b that converges toward the front-end protrusion 158a along a diagonal direction following both the major-axis direction Z1 and the minor-axis direction Z2, thereby connecting the differential widths between the wide rear end 158c and the narrow front-end protrusion 158a. In one embodiment of the present invention, the rear end 158c defining the boundary of the assembly guide unit 158 may represent the boundary portion of the assembly guide unit 158 having a wide width corresponding to the full width W₀ of the optically visible iris-pupil complex expansion part 105. Here, the full width W₀ of the optically visible iris-pupil complex expansion part 105 refers to the complete width of the optically visible iris-pupil complex expansion part 105 in which no assembly slit 156a, such as the assembly slit 156a introduced from the inner annular edge 101 and/or outer annular edge 102, is formed, for example, representing a region of the optically visible iris-pupil complex expansion part 105 having a wider width than the neck W₁, which has a narrow width defined by the assembly slit 156a.

In one embodiment of the present invention, the assembly guide unit 158 may be formed on the front side of the first end E₁ along the assembly direction or major-axis direction Z1 of the first and second ends E₁ and E₂. An assembly slit 156a may be formed on the rear side of the first end E₁. In another embodiment of the present invention, a relatively wide portion of the first end E₁ may be interposed between the front-side assembly guide unit 158 and the rear-side assembly slit 156a. For example, the relatively wide portion of the first end E₁ interposed between the assembly guide unit 158 and the assembly slit 156a may correspond to the full width W₀ of the optically visible iris-pupil complex expansion part 105, which has a relatively wide and continuous width where no assembly slit 156a introduced from the inner annular edge 101 and/or the outer annular edge 102 is formed.

For example, in one embodiment of the present invention, the assembly slit 156a may be formed in a shape drawn inward from the inner annular edge 101 and the outer annular edge 102 toward the interior of the optically visible iris-pupil complex expansion part 105 along the minor-axis direction Z2. In this manner, the width of the neck W₁, which defines the minimum width while being drawn inward from both the inner annular edge 101 and the outer annular edge 102 that define the optically visible iris-pupil complex expansion part 105, may be equal to or smaller than the width W₂ of the assembly hole 156b on the second end E₂. In one embodiment of the present invention, the assembly slit 156a on the first end E₁ may be arranged in plurality along the major-axis direction Z1 or along the length of the first end E₁. For example, in one embodiment of the present invention, through the arrangement of the assembly slits 156a formed along the major-axis direction Z1 or along the length of the first end E₁, the binding position between the first and second ends E₁ and E₂ may be varied so as to form a circumferential length optimized for surrounding the optically visible iris-pupil complex of the implant recipient.

In one embodiment of the present invention, the assembly guide unit 158 on the first end E₁ may align the binding positions between the first and second ends E₁ and E₂ and guide the assembly slit 156a, formed behind the assembly guide unit 158 of the first end E₁, toward the assembly hole 156b on the second end E₂ side. In one embodiment of the present invention, the width of the front-end protrusion 158a formed in the assembly guide unit 158 of the first end E₁ may be smaller than the width W₂ of the assembly hole 156b on the second end E₂. When the front-end protrusion 158a of the first end E₁ is fitted into the assembly hole 156b of the second end E₂, the binding positions of the first and second ends E₁ and E₂ may be aligned with each other. When the operator of the ocular implant 100 pulls the front-end protrusion 158a of the first end E₁, which is fitted into the assembly hole 156b of the second end E₂, toward the second end E₂, the relatively wide portion of the first end (E₁, full width W₀ of the first end E₁) connected to the variable-width portion 158b of the assembly guide unit 158 may be guided toward the assembly hole 156b on the second end E₂. At this time, when the assembly hole 156b of the second end E₂ is fitted into the neck W₁ of the assembly slit 156a that is continuous with the wide portion (E₁, full width W₀ of the first end E₁), separation between the first and second ends E₁ and E₂ can be prevented. For example, the relatively wide portion of the first end (E₁, full width W₀ of the first end E₁) positioned in front of and behind the neck W₁ of the assembly slit 156a, which is fitted into the assembly hole 156b of the second end E₂, may function as a catch or hook, thereby preventing disengagement between the first and second ends E₁ and E₂.

In one embodiment of the present invention, the front assembly guide unit 158 is configured to guide the catch-engaging or hook-engaging connection between the neck W₁ of the rear assembly slit 156a and the assembly hole 156b, by allowing the relatively thick first end (E₁, full width W₀ of the first end E₁) positioned at the front to pass rearward through the assembly guide unit 158 along the assembly direction or major-axis direction Z1, and then through the assembly hole 156b, which is formed with a relatively narrow width. In the case of the assembly guide unit 158, once the first and second ends E₁ and E₂ are bound to each other through the catch-engaging or hook-engaging connection, that is, after the neck W₁ of the assembly slit 156a of the first end E₁ is fitted into the assembly hole 156b of the second end E₂, the assembly guide unit 158, having served its purpose, may be removed. For example, by removing the assembly guide unit 158 that overlaps the second end E₂ and forms an additional thickness, adverse effects on the surrounding tissues of the eyeball EB can be prevented, and deformation of the ocular implant 100 can be avoided.

In one embodiment of the present invention, a cut line CL may be formed along the rear boundary corresponding to the rear end 158c of the assembly guide unit 158 in the assembly direction or major-axis direction Z1. After the first and second ends E₁ and E₂ are bound to each other through a catch-engaging or hook-engaging connection, the assembly guide unit 158 may be separated and removed along the cut line CL. The cut line CL may be formed between the front-side assembly guide unit 158 and the rear-side assembly slit 156a, and more specifically, may be formed along the boundary (i.e., the rear boundary of the assembly guide unit 158 forming its rear end 158c) between the front-side assembly guide unit 158 and the relatively wide portion of the first end (E₁, full width W₀ of the first end E₁) positioned between the front-side assembly guide unit 158 and the rear-side assembly slit 156a. The cut line CL may include a plurality of perforations formed along the cut line CL, or alternatively, may be provided as an indication line marking the cutting position in a non-perforated form. In one embodiment of the present invention, the cut line CL may be formed parallel to the incision C along the minor-axis direction Z2.

In various embodiments of the present invention, the first and second ends E₁ and E₂ separated from each other across the incision C may be joined by any of the following means: (i) by using the surgical holes 10 formed in the first and second ends E₁ and E₂ and passing a suture therethrough to bind the first and second ends E₁ and E₂ together; (ii) by using the first and second slits 154a, 154b, 155a and 155b formed in the first and second ends E₁ and E₂ and engaging the first and second ends E₁ and E₂ with each other through the first and second slits 154a, 154b, 155a and 155b to achieve binding between the first and second ends E₁ and E₂; or (iii) by using the assembly slit 156a and the assembly hole 156b formed in the first and second ends E₁ and E₂ so that the assembly slit 156a on the first-end E₁ side is fitted into the assembly hole 156b on the second-end E₂ side to bind the first and second ends E₁ and E₂ together. In this way, in various embodiments of the present invention, during the binding of the first and second ends E₁ and E₂, the first and second ends E₁ and E₂ to be bound may either face each other along the assembly direction or major-axis direction Z1 without overlapping and be bound in a face-to-face manner, or the first and second ends E₁ and E₂ may overlap each other to form the binding structure. As illustrated in FIGS. 10A to 11B, in a configuration in which the first and second ends E₁ and E₂ are provided with the first and second slits 154a, 154b, 155a and 155b and are fitted to each other, or in a configuration in which the first and second ends E₁ and E₂ are respectively provided with the assembly slit 156a and the assembly hole 156b and are fitted to each other, the first and second ends E₁ and E₂ may overlap each other and form an extra thickness. Such an extra thickness locally generated along the circumferential direction of the ocular implant 100 or of the optically visible iris-pupil complex expansion part 105 forming the body of the ocular implant 100 may impose localized stress or deformation. Therefore, in consideration of such factors, among the first and second ends E₁ and E₂, in the overlapping region between the first and second slits 154a, 154b, 155a and 155b and the incision C, or in the overlapping region between the neck W₁ of the assembly slit 156a, the assembly hole 156b, and the incision C, that is, except for (i) a portion of the first end E₁ that is formed in front of the neck W₁ of the assembly slit 156a, which is inserted into the assembly hole 156b, defines the full width W0 of the optically visible iris-pupil complex expansion part 105, and functions as a catch or hook, and (ii) a portion of the second end E₂ formed in front of the assembly hole 156b to maintain the closed shape of the assembly hole 156b, the remaining overlapping regions where the first and second ends E₁ and E₂ overlap each other are formed with a relatively thin thickness. Accordingly, in the ocular implant 100 after transplantation, or in the ocular implant 100 in which the binding between the first and second ends E₁ and E₂ has been completed, a uniform thickness can be maintained over the entire circumference surrounding the outer ring of the optically visible iris-pupil complex of the implant recipient. For example, as shown in FIGS. 10A to 11B, around the first and second slits 154a, 154b, 155a and 155b formed on the first and second ends E₁ and E₂, thin-walled portions formed with a relatively reduced thickness may be provided so that additional excess thickness due to overlapping of the first and second ends E₁ and E₂ is not generated. The thin-walled portions of the first and second ends E₁ and E₂ may overlap each other to form the normal design thickness of the ocular implant 100.

In one embodiment of the present invention, the catch-engaging or hook-engaging connection structure that binds the first and second ends E₁ and E₂, which are separated from each other through the incision C, toward each other, may be formed at a relatively wide position among the positions on both sides along the minor-axis direction Z2. By being formed at a position extending generally along the major-axis direction Z1 where the curvature is relatively small, the engagement between the first and second ends E₁ and E₂ can be achieved more easily during the implantation procedure of the ocular implant 100. This one-side position may correspond to the central position S of the implantation procedure.

Throughout this specification, the central position S of the implantation procedure is described as a position that is relatively wide among the one-side and the other-side positions of the optically visible iris-pupil complex expansion part 105, which define both sides along the minor-axis direction Z2, where the elliptical profile of the inner annular edge 101 is deflected toward the opposite side within the circular profile of the outer annular edge 102. However, in the embodiments illustrated in the drawings attached to the present specification, the central position S of the procedure may be selectively formed at either one of the one-side position (0-degree angular position A1) and the other-side position (180-degree angular position A2), where the elliptical profile of the inner annular edge 101 is symmetrically positioned within the circular profile of the outer annular edge 102 along the minor-axis direction Z2 without being biased toward either side, and the two positions have substantially the same width. In one embodiment of the present invention, the one-side position forming the central position S of the procedure may be selected from among the two positions along the minor-axis direction Z2, thereby forming a relatively small curvature that extends generally along the major-axis direction Z1.

In the present specification, with respect to the arrangement of the plurality of surgical holes 10 formed along the first and second ends E₁ and E₂ separated from each other through the incision C, the plurality of first and second slits 154a, 154b, 155a and 155b formed along the first and second ends E₁ and E₂ to form a catch-engaging or hook-engaging connection, or the assembly slits 156a formed along the first and second ends E₁ and E₂, the arrangement of these structures may be along the length of the first and second ends E₁ and E₂. For example, they may be arranged along the length of the first and second ends E₁ and E₂ that form a portion of the circumference of the optically visible iris-pupil complex expansion part 105. Accordingly, throughout this specification, when these arrays are described as being arranged along the major-axis direction Z1, it may mean that they are arranged along the circumferential direction of the optically visible iris-pupil complex expansion part 105.

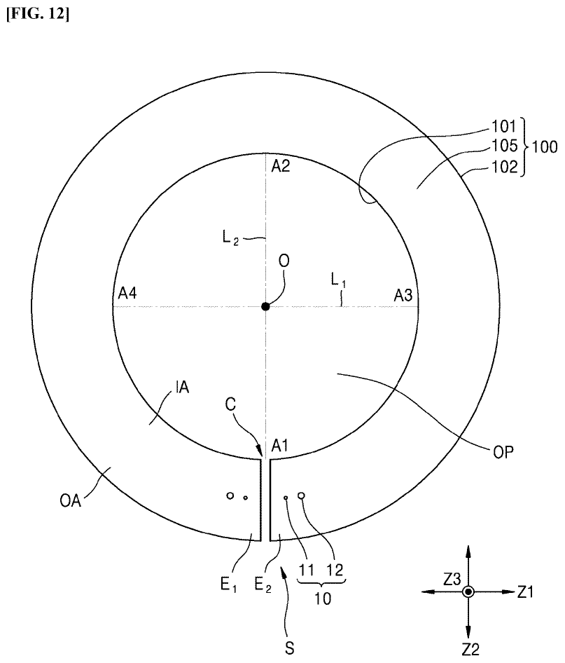

FIG. 12 illustrates a modified embodiment of the ocular implant shown in FIG. 4. Referring to FIG. 12, the outer annular edge 102 may be formed in a circular profile, and the inner annular edge 101 having a circular profile may be formed inside the circular profile of the outer annular edge 102. For example, in one embodiment of the present invention, the inner annular edge 101 and the outer annular edge 102 may surround the opening OP with the same circular profile. They may surround the opening OP in a concentric configuration with the center O of the opening OP as the center of the circles. In one embodiment of the present invention, the inner annular edge 101 may be formed in a circular shape in which the major axis length L₁, extending in the direction in which a pair of the implant recipient's eyes face each other, and the minor axis length L₂, perpendicular to the major axis length L₁, are equal to each other. By forming the inner annular edge 101 with equal major axis length L₁ and minor axis length L₂, so that alignment with the implant recipient's optically visible iris-pupil complex is unnecessary, the convenience of the procedure can be improved. Furthermore, since the outer annular edge 102 and the inner annular edge 101 are formed in a concentric circular shape with the center O of the opening OP as the centroid, the convenience of the machining process for forming the ocular implant 100 can also be improved.

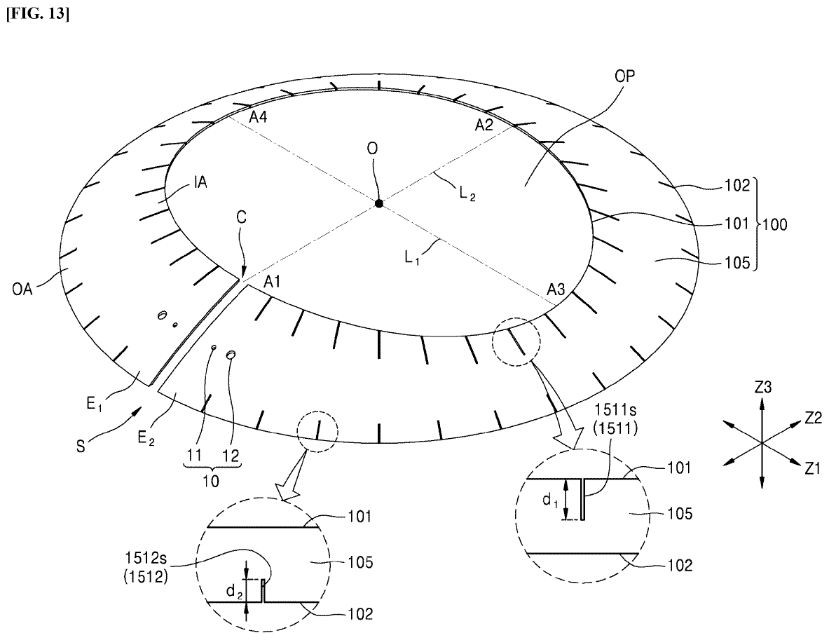

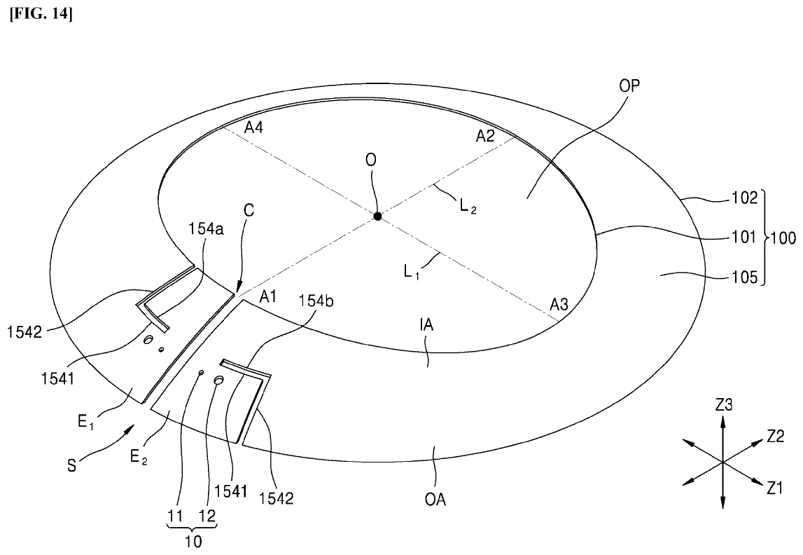

FIG. 13 is a view illustrating a modified embodiment of the ocular implant shown in FIG. 6. Referring to FIG. 13, the adaptive stretchable parts 1511 and 1512 can provide longitudinal stretching along the peripheries of the inner annular edge 101 and the outer annular edge 102, allowing adaptive deformation according to the shape and size of the optically visible iris-pupil complex of each implant recipient. For example, the adaptive stretchable part 1511 of the inner annular edge 101 may include a plurality of slits 1511s formed to be spaced apart from each other along the perimeter of the inner annular edge 101. Similarly, the adaptive stretchable part 1512 of the outer annular edge 102 may include a plurality of slits 1512s formed to be spaced apart from each other along the perimeter of the outer annular edge 102. In one embodiment of the present invention, the width d₁ of each slit 1511s forming the adaptive stretchable part 1511 of the inner annular edge 101 may be defined along the depth direction extending from the inner annular edge 101 to the outer annular edge 102. The width d₂ of each slit 1512s forming the adaptive stretchable part 1512 of the outer annular edge 102 may be defined along the depth direction extending from the outer annular edge 102 toward the inner annular edge 101. In one embodiment of the present invention, the width d₂ of the slits 1152s forming the adaptive stretchable part 1152 of the outer annular edge 102 may be smaller than the width d₁ of the slits 1151s forming the adaptive stretchable part 1151 of the inner annular edge 101. For example, the sizes of the slit widths d₁ and d₂ forming the respective adaptive stretchable parts 1151 and 1152 may be adjusted such that the range of longitudinal stretching provided by the inner annular edge 101 is wider than that provided by the outer annular edge 102. In one embodiment of the present invention, for the adaptive stretchable parts 1511 and 1512, it is preferable that the range of longitudinal stretching of the inner annular edge 101, which surrounds the optically visible iris-pupil complex at a relatively close position, is wider than the range of longitudinal stretching of the outer annular edge 102, which surrounds the optically visible iris-pupil complex at a relatively distant position, thereby allowing adaptive deformation to the individual anatomical characteristics of each implant recipient's optically visible iris-pupil complex. To this end, the sizes of the slit widths d₁ and d₂ forming the respective adaptive stretchable parts 1151 and 1152 may be appropriately controlled. In various embodiments of the present invention, the adaptive stretchable parts 1511 and 1512 may be formed along either the perimeter of the inner annular edge 101 or the outer annular edge 102, or may be formed on both sides, that is, along both the inner annular edge 101 and the outer annular edge 102.

FIG. 14 is a view illustrating a composite configuration of different mechanisms for connecting the first and second ends E₁ and E₂, which are separated from each other through the incision C, toward each other, according to one embodiment of the present invention. In the ocular implant 100 according to one embodiment shown in the figure, the surgical hole 10 and a catch-engaging or hook-engaging connection structure may be formed in combination. Referring to FIG. 14, in the ocular implant 100 according to one embodiment of the present invention, first and second slits 154a and 154b may be formed on the sides of the first and second ends E₁ and E₂, respectively, as the catch-engaging or hook-engaging connection structure, together with the surgical hole 10. Referring also to FIG. 11A, in another embodiment of the present invention, the ocular implant 100 may include, together with the surgical hole 10, an assembly slit 156a and an assembly hole 156b formed on the sides of the first and second ends E₁ and E₂ as the catch-engaging or hook-engaging connection structure.

In this way, in one embodiment of the present invention, a catch-engaging or hook-engaging connection structure is formed in combination with the surgical hole 10, which allows insertion of a suture to bind the first and second ends E₁ and E₂ to each other through a suture knot, together with another mechanism for coupling the first and second ends E₁ and E₂ to each other. Accordingly, for example, by combining different mechanisms for coupling the first and second ends E₁ and E₂ to each other, the coupling strength between the first and second ends E₁ and E₂ can be enhanced. For instance, if it is determined by the surgeon that sufficient coupling strength can be achieved between the first and second ends E₁ and E₂ only by the catch-engaging or hook-engaging connection, the suturing through the surgical hole 10 may be omitted. Alternatively, when the first and second ends E₁ and E₂ are temporarily fastened by the catch-engaging or hook-engaging connection, the coupling strength between the first and second ends E₁ and E₂ may be reinforced by suturing through the surgical hole 10, thereby firmly securing the first and second ends E₁ and E₂ to each other. For example, in one embodiment of the present invention, the surgical hole 10, formed in combination with the catch-engaging or hook-engaging connection, may include the first surgical hole 11 for the passage of a suture, and the second surgical hole 12 for physical engagement with or passage of an implant insertion device, which provide a binding position between the first and second ends E₁ and E₂.

Throughout this specification, as a bonding mechanism for joining the first and second ends E₁ and E₂, which are separated from each other through the incision C, toward each other, in addition to the suture passing through the surgical hole 10 or the catch-engaging or hook-engaging connection, the first and second ends E₁ and E₂ may be arranged to overlap each other, and a bio-adhesive may be applied between the first and second ends E₁ and E₂. Alternatively, the first and second ends E₁ and E₂ may be arranged to overlap each other, and one of the first and second ends E₁ and E₂ may be thermally fused to the other of the first and second ends E₁ and E₂ by heat melting. In the fusion bonding of the first and second ends E₁ and E₂, the first and second ends E₁ and E₂ may be softened or tackified through a high heat input and then pressed toward each other so that the first and second ends E₁ and E₂ are mutually bonded. In other words, in various embodiments of the present invention, various mechanisms for bonding the first and second ends E₁ and E₂ to each other may be provided either individually or in combination with different mechanisms. For example, when a bonding method such as bio-adhesive application or thermal fusion as described above is applied, the surgical hole 10 or the first and second slits 154a and 154b for a catch-engaging or hook-engaging connection, or the assembly slit 156a or assembly hole 156b, may not be formed on the first and second ends E₁ and E₂.

In one embodiment of the present invention, the ocular implant 100 is configured such that the optically visible iris-pupil complex expansion part 105 disposed between the inner annular edge 101 and the outer annular edge 102 is formed so that one side position along the minor-axis direction of the inner annular edge 101 has a wider width than the other side position. Accordingly, the elliptical or circular profile of the inner annular edge 101 may be formed in a biased position, shifted toward the other side, within the circular profile of the outer annular edge 102. As a result, the one-sided position of the optically visible iris-pupil complex expansion part 105 formed with a relatively wider width among the two side positions along the minor-axis direction of the inner annular edge 101 can provide the center position O of the procedure.

However, in one embodiment of the present invention, within the circular profile of the outer annular edge 102, the elliptical or circular profile of the inner annular edge 101 may be formed such that the inner annular edge 101 has equal widths without being biased toward either one or the other side along the minor-axis direction. In such a case, surgical inconvenience may arise for the practitioner, who must identify a relatively wide position along the minor-axis direction to align the center position O of the ocular implant 100 during implantation. Additionally, during the molding process of the ocular implant 100, handling difficulties may occur due to insufficient rigidity at one or the other side position having a relatively narrow width in a biased configuration. Accordingly, in one embodiment of the present invention, the ocular implant 100 may be formed such that both side positions along the minor-axis direction have substantially equal widths, without being biased toward either side. In other words, the inner annular edge 101 may be formed in a symmetrical position within the circular profile of the outer annular edge 102, such that both side positions are balanced along the minor-axis direction. Meanwhile, in various embodiments of the present invention, the inner annular edge 101 may be formed into either an elliptical or circular profile. In an elliptical configuration, the major axis length L₁ and the minor axis length L₂ are different from each other, whereas in a circular configuration, where the major axis length L₁ and the minor axis length L₂ are equal, the major axis may represent the direction in which a pair of the implant recipient's eyes face each other, and the minor axis may represent the direction perpendicular to the major axis.

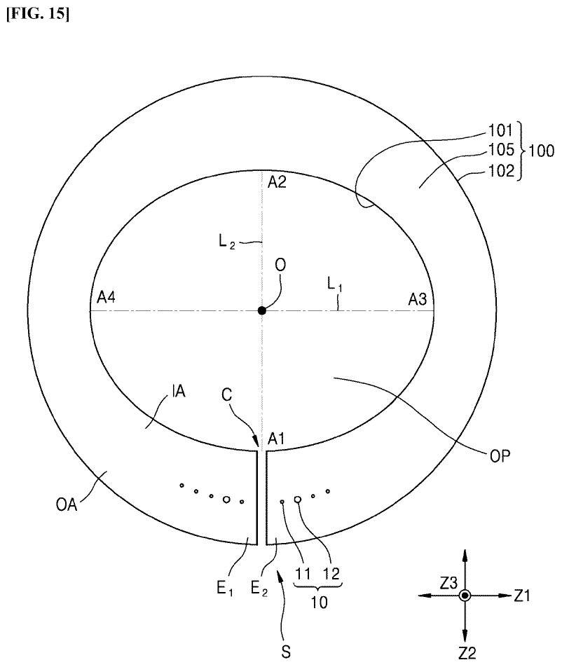

FIG. 15 is a view illustrating a modified embodiment of the ocular implant 100 shown in FIG. 9. Referring to FIG. 15, in one embodiment of the present invention, the surgical holes 10 formed on the first and second ends E₁ and E₂, which are separated from each other through the incision C, may be provided in asymmetric numbers. Accordingly, a surgeon performing implantation of the ocular implant 100 can recognize and confirm the orientation of the ocular implant 100 based on the arrangement of the surgical holes 10 formed on the first and second ends E₁ and E₂. For example, in one embodiment of the present invention, during implantation of the ocular implant 100, the implant 100 may be pulled along the outer ring of the optically visible iris-pupil complex through the second surgical hole 12 of the surgical holes 10 fitted with a transplant insertion device configured to insert the ocular implant 100 so as to circumferentially travel around the implant recipient's optically visible iris-pupil complex. The direction in which the ocular implant 100 is pulled along the outer ring of the optically visible iris-pupil complex through the transplant insertion device may be predetermined according to the structural design of the insertion device. Accordingly, after the ocular implant 100 has been correctly recognized and aligned by the surgeon in the proper orientation, the transplant insertion device must be inserted into the second surgical hole 12 of the surgical holes 10 and operated so that it travels circumferentially along the outer ring of the optically visible iris-pupil complex in the correct direction. In one embodiment of the present invention, since the ocular implant 100 may be formed of a soft, biocompatible material such as silicone, which can flexibly and adaptively deform on the tissues of the eyeball EB while being harmless thereto, the anterior curved surface 120 and the posterior inclined surface 110 of the ocular implant 100 may be flipped over each other. Even when such inversion occurs, that is, when the ocular implant 100 is flipped so that the anterior curved surface 120, instead of the posterior inclined surface 110, faces the eyeball EB, and the posterior inclined surface 110 faces outward toward the external space OS opposite to the eyeball EB, the surgeon may find it difficult to easily detect this orientation error during the procedure. Therefore, according to one embodiment of the present invention, the ocular implant 100 is designed such that the surgical holes 10 formed on the first and second ends E₁ and E₂, which are separated from each other through the incision C, are provided in an asymmetrical number. For instance, the number of surgical holes 10 on the first and second ends E₁ and E₂ may be different, or alternatively, the surgical holes 10 formed on the first and second ends E₁ and E₂ may be arranged such that one of the first and second ends E₁ and E₂ includes an even number of surgical holes 10 while the other of the first and second ends E₁ and E₂ includes an odd number of surgical holes 10. Accordingly, when the surgeon, referring to the structural design of the transplant insertion device, identifies which of the first and second ends E₁ and E₂ is to receive the insertion device, if the expected total number, expected odd number, or expected even number of surgical holes 10 formed on the first and second ends E₁ and E₂ is not observed, the surgeon can immediately recognize that the corresponding ocular implant 100 has been inverted, that is, the anterior curved surface 120, which is intended to face the external space OS opposite to the eyeball EB, and the posterior inclined surface 110, which is intended to face toward the eyeball EB, have been reversed with respect to each other. Consequently, the erroneous orientation of the ocular implant 100 can be corrected, and the implant 100 can be properly realigned so that the anterior curved surface 120 faces the external space OS opposite to the eyeball EB and the posterior inclined surface 110 faces the eyeball EB, as intended by the design of the implant. For example, in various embodiments of the present invention, the orientation of the ocular implant 100 may be confirmed through an asymmetrical design in which the number of surgical holes 10 formed on the first and second ends E₁ and E₂ separated by the incision C differs from each other, or in which the surgical holes 10 formed on the first and second ends E₁ and E₂ are even on one end and odd on the other, thereby enabling the surgeon to easily verify the correct orientation of the ocular implant 100 during surgery.

While the present invention has been described with reference to the embodiments illustrated in the accompanying drawings, these embodiments are merely exemplary, and it will be understood by those skilled in the art that various modifications, equivalent structures, and alternative embodiments may be made without departing from the scope of the present invention.

### Industrial Applicability

The present invention can be applied to the medical industry, particularly to the manufacture of ocular implants for surgical procedures and to the production of surgical instruments associated therewith.

1. An ocular implant permanently implanted on the eyeball of an implant recipient to provide an enlarged appearance of the implant recipient's optically visible iris-pupil complex, comprising:
a central opening configured to receive light incident on the optically visible iris-pupil complex that provides an optical aperture of the eyeball;
an inner annular edge surrounding and defining the opening;
an outer annular edge disposed opposite the inner annular edge that defines the opening and surrounds the opening together with the inner annular edge; and
an optically visible iris-pupil complex expansion part formed with a variable thickness that changes from the inner annular edge to the outer annular edge between the inner annular edge and the outer annular edge.

2. The ocular implant according to claim 1, wherein the optically visible iris-pupil complex expansion part comprises:
a posterior inclined surface disposed to face the eyeball so as to closely adhere to the eyeball; and
an anterior curved surface disposed toward an external space opposite to the eyeball.

3. The ocular implant according to claim 2, wherein the inner annular edge and the outer annular edge each form an anterior portion of the eyeball and appear as an ellipse and a circle when viewed along a frontal direction from the anterior portion of the eyeball, where a crystalline lens that performs a lens function and a pupil forming a light entrance aperture are located, toward a posterior portion of the eyeball,
wherein the posterior inclined surface is formed at a constant tilting angle from a vertical plane perpendicular to the frontal direction, such that the inner annular edge and the outer annular edge define an anterior position and a posterior position, respectively.

4. The ocular implant according to claim 2, wherein the posterior inclined surface provides a support surface that is supported by an inclined surface formed in a packaging container of the ocular implant.

5. The ocular implant according to claim 2, wherein the anterior curved surface is formed along a spline curve whose curvature or radius of curvature varies along the inclination direction of the posterior inclined surface.

6. The ocular implant according to claim 2, wherein the optically visible iris-pupil complex expansion part has a variable thickness such that the thickness between the posterior inclined surface and the anterior curved surface varies.

7. The ocular implant according to claim 6, wherein the thickness of the optically visible iris-pupil complex expansion part is measured along a direction perpendicular to the posterior inclined surface, from the posterior inclined surface to the anterior curved surface.

8. The ocular implant according to claim 2, wherein the inner annular edge is formed by contact between the anterior curved surface and the posterior inclined surface, which define the thickness of the optically visible iris-pupil complex expansion part, at a position adjacent to the opening.

9. The ocular implant according to claim 2, wherein the outer annular edge is formed by contact between the anterior curved surface and the posterior inclined surface, which define the thickness of the optically visible iris-pupil complex expansion part, at a position opposite to the inner annular edge defining the opening.

10. The ocular implant according to claim 2, wherein the anterior curved surface and the posterior inclined surface, which define the thickness of the optically visible iris-pupil complex expansion part, contact each other to form the inner annular edge and the outer annular edge, each having a rounded edge shape.

11. The ocular implant according to claim 10, wherein the inner annular edge is formed with a relatively gently rounded edge shape, and
the outer annular edge is formed with a relatively sharply rounded edge shape.

12. The ocular implant according to claim 11, wherein the thickness at the edge of the inner annular edge is smaller than the thickness at the edge of the outer annular edge.

13. The ocular implant according to claim 11, wherein the inner annular edge extends to an irregular edge between a cornea having a large-curvature portion that convexly protrudes forward and a sclera having a small-curvature portion extending posteriorly from the cornea, among the membrane tissues surrounding the entire eyeball to maintain its shape, and the inner annular edge has a relatively gently rounded edge shape that defines one side of the ocular implant,
wherein the outer annular edge, which closely adheres to the sclera in the large-curvature portion, has a relatively sharply rounded edge shape that defines the opposite side of the ocular implant, so as to prevent lifting from the sclera or the formation of a gap between the sclera and the outer annular edge.

14. The ocular implant according to claim 1, wherein the optically visible iris-pupil complex expansion part comprises:
a maximum-thickness portion having a maximum thickness between the inner annular edge and the outer annular edge;
an inner area formed relatively close to the opening and disposed between the inner annular edge and the maximum-thickness portion; and
an outer area formed relatively far from the opening and disposed between the maximum-thickness portion and the outer annular edge.

15. The ocular implant according to claim 14, wherein the inner area and the outer area disposed on both sides of the maximum-thickness portion have an asymmetrical shape or an asymmetrical thickness profile.

16. The ocular implant according to claim 14, wherein the inner area is formed of a relatively soft material so as to adaptively deform to fit the optically visible iris-pupil complex of each implant recipient, and
the outer area is formed of a relatively rigid material so as to maintain a smoothly circular appearance against deformation of the inner area and to reinforce the flexibility of the inner area, thereby providing support rigidity of the ocular implant.

17. The ocular implant according to claim 16, wherein the inner area is formed with a thinner thickness than the outer area, the inner area extending to an irregular edge between a cornea having a large-curvature portion that convexly protrudes forward and a sclera having a small-curvature portion extending posteriorly from the cornea, among the membrane tissues surrounding the entire eyeball to maintain its shape and adhering closely to the irregular edge so as to prevent the formation of a gap between the irregular edge and the inner area.

18. The ocular implant according to claim 16, wherein the inner area and the outer area are formed of the same material, and
the inner area is formed with a relatively smaller thickness than the outer area.

19. The ocular implant according to claim 18, wherein the optically visible iris-pupil complex expansion part includes an anterior curved surface and a posterior inclined surface that define the thickness of the optically visible iris-pupil complex expansion part, and
wherein a first average thickness of the inner area and a second average thickness of the outer area, formed by the anterior curved surface and the posterior inclined surface, satisfy the relationship that first average thickness<second average thickness.

20. The ocular implant according to claim 18, wherein, based on the posterior inclined surface providing a reference for the thickness of the optically visible iris-pupil complex expansion part,
an anterior curved surface forming the inner area extends along a trajectory relatively close to the posterior inclined surface, and an anterior curved surface forming the outer area extends along a trajectory relatively farther from the posterior inclined surface, thereby forming a difference in thickness between the inner area and the outer area disposed on both sides of the maximum-thickness portion.

21. The ocular implant according to claim 20, wherein the anterior curved surface forming the inner area extends along a trajectory relatively close to the posterior inclined surface, forming a relatively thin thickness, and then forms a gently rounded edge at the inner annular edge along a relatively gentle downward curve toward the posterior inclined surface, and
wherein the anterior curved surface forming the outer area extends along a trajectory relatively farther from the posterior inclined surface, forming a relatively greater thickness, and then forms a sharply rounded edge at the outer annular edge along a relatively steep downward curve toward the posterior inclined surface.

22. An ocular implant permanently implanted on the eyeball of an implant recipient to provide an enlarged appearance of the implant recipient's optically visible iris-pupil complex, comprising:
a central opening configured to receive light incident on the optically visible iris-pupil complex that provides an optical aperture of the eyeball;
an inner annular edge surrounding and defining the opening;
an outer annular edge disposed opposite the inner annular edge that defines the opening and surrounds the opening together with the inner annular edge; and
an adaptive stretchable part configured to provide adaptive longitudinal stretching along the perimeter of at least one of the inner annular edge and the outer annular edge.

23. The ocular implant according to claim 22, wherein the adaptive stretchable part provides longitudinal stretching along the perimeter of the inner annular edge so as to adaptively deform to fit the shape and size of the optically visible iris-pupil complex of each implant recipient.

24. The ocular implant according to claim 22, wherein the adaptive stretchable part includes at least one slit formed along the perimeter of the inner annular edge.

25. The ocular implant according to claim 24, wherein the adaptive stretchable part includes a plurality of slits formed to be spaced apart from each other along the perimeter of the inner annular edge.

26. The ocular implant according to claim 25, wherein the adaptive stretchable part includes a group of slits formed at opposite positions along a major axis and/or at opposite positions along a minor axis.

27. The ocular implant according to claim 26, wherein the adaptive stretchable part comprises:
first and second groups of slits formed at opposite positions along the minor axis, through which divided segments overlap each other to extend the minor axis length while shortening the major axis length; and
third and fourth groups of slits formed at opposite positions along the major axis, through which the divided segments overlap each other to extend the major axis length while shortening the minor axis length.

28. The ocular implant according to claim 24, wherein the optically visible iris-pupil complex expansion part comprises:
a posterior inclined surface disposed to face the eyeball so as to closely adhere to the eyeball; and
an anterior curved surface disposed toward the external space opposite to the eyeball and forming a thickness profile based on the posterior inclined surface, wherein the anterior curved surface contacts the posterior inclined surface at a position adjacent to the opening to form an inner annular edge, and also contacts the posterior inclined surface at a position opposite the opening to form an outer annular edge, and forms a thickness profile based on the posterior inclined surface to provide a relatively thin inner area and a relatively thick outer area based on a maximum-thickness portion that defines the maximum thickness between the inner annular edge and the outer annular edge; and
wherein the divided segments formed through the slits constituting the adaptive stretchable part overlap each other to expand the length of the inner annular edge, thereby forming an additional thickness that increases the thickness of the inner area into which the slit extends from the inner annular edge.

29. The ocular implant according to claim 1, wherein the inner annular edge, at which the divided segments formed through the slits constituting the adaptive stretchable part overlap each other, is formed with a relatively gently rounded edge, and
the outer annular edge is formed with a relatively sharply rounded edge.

30. An ocular implant permanently implanted on the eyeball of an implant recipient to provide an enlarged appearance of the implant recipient's optically visible iris-pupil complex, comprising:
a central opening configured to receive light incident on the optically visible iris-pupil complex that provides an optical aperture of the eyeball;
an inner annular edge surrounding and defining the opening;
an outer annular edge disposed opposite the inner annular edge that defines the opening and surrounds the opening together with the inner annular edge; and
an optically visible iris-pupil complex expansion part formed between the inner annular edge and the outer annular edge, the optically visible iris-pupil complex expansion part including first and second ends that are separated from each other through an incision and are joined to each other through respective surgical holes formed therein.

31. The ocular implant according to claim 30, wherein the surgical holes comprise:
a first surgical hole through which a suture passes to connect the first and second ends to each other; and
a second surgical hole through which an implantation insertion tool passes to pull the ocular implant so that the ocular implant travels along the outer ring of the implant recipient's optically visible iris-pupil complex through an incision in a conjunctiva on the sclera.

32. The ocular implant according to claim 31, wherein the first surgical hole is formed with a relatively small diameter, and
the second surgical hole is formed with a relatively large diameter.

33. The ocular implant according to claim 31, wherein the first surgical hole is formed at a position relatively close to the incision, and
the second surgical hole is formed at a position relatively farther from the incision.

34. An ocular implant permanently implanted on the eyeball of an implant recipient to provide an enlarged appearance of the implant recipient's optically visible iris-pupil complex, comprising:
a central opening configured to receive light incident on the optically visible iris-pupil complex that provides an optical aperture of the eyeball;
an inner annular edge surrounding and defining the opening;
an outer annular edge disposed opposite the inner annular edge that defines the opening and surrounds the opening together with the inner annular edge; and
an optically visible iris-pupil complex expansion part formed between the inner annular edge and the outer annular edge, the optically visible iris-pupil complex expansion part including first and second ends that are separated from each other through an incision and form a catch-engaging or hook-engaging connection with each other.
